(19) United States
(12) Patent Application Publication
(10) Pub. No.: US 2010/0104564 A1
(43) Pub. Date: Apr. 29, 2010

(54) Altered Antibody Fc Regions and Uses Thereof

(76) Inventors: Genevieve Hansen, Rancho Santo Fe, CA (US); Gerald Neslund, Lynnwood, WA (US); Amelia Black, Los Gatos, CA (US); Josephine Cardarelli, San Carlos, CA (US); Srinivasan Mohan, Cupertino, CA (US); David J. King, Solana Beach, CA (US)

Correspondence Address: BAKER BOTTS L.L.P., 30 ROCKEFELLER PLAZA, 44th Floor, NEW YORK, NY, 10112-4498, US

(21) Appl. No.: 11/887,467

(22) PCT Filed: Mar. 28, 2006
(86) PCT No.: PCT/US2006/011048

§ 371 Date: Dec. 15, 2009

Related U.S. Application Data
(60) Provisional application No. 60/666,010, filed on Mar. 29, 2005.

(51) Int. Cl. A61K 39/395; C07K 16/00; A61P 3/00; A61P 35/00; A61P 9/00; A61P 17/00; A61P 31/00; A61P 19/10; A61P 1/00; C12P 21/00; G01N 33/53

(52) U.S. Cl. 424/133.1; 530/387.3; 435/71.1; 436/501

(57) ABSTRACT

The present invention relates to altered antibody Fc regions and uses thereof.

### CROSS REFERENCE TO RELATED APPLICATIONS

The present application claims the benefit of the filing date of U.S. application Ser. No. 60/666,010, filed on Mar. 29, 2005 under 35 U.S.C. §119(e), the disclosure of which is incorporated by reference herein.

### FIELD OF THE INVENTION

The present invention relates to altered antibody Fc regions and uses thereof.

### BACKGROUND

Monoclonal antibodies (mAb) are unique and versatile molecules that have found applications in research, diagnosis, and in the treatment of multiple diseases, including cancer. The advent of hybridoma technology for monoclonal antibody production in 1975 was a breakthrough in the field of biomedicine; at least 17 of them have FDA approval for therapeutic use in patients.

The use of molecular biological techniques allows for the construction of chimeric antibodies with both human and murine elements. These chimeric antibodies have a mouse-derived variable antigen-specific region fused to a heavy chain derived from humans. Moreover, the use of phage display, transgenic mice and mutagenesis allow for the selection and identification of fully human antibodies, as well as selection of improvements in antibody affinity, avidity and pharmacokinetics. The ability to generate human monoclonal antibodies achieved 2 important goals: it overcame most host anti-antibody responses, and it extended the half-life of the reagent.

Other strategies to improve antibody properties that alter antibody structure include increasing the molecular weight of the molecule to above the renal threshold or altering surface charge, which provides for increased circulating half-life.

However, there is a continuing need for improved antibodies.

### SUMMARY OF THE INVENTION

Certain embodiments of the present invention pertain to altered Fc regions of antibodies, and uses thereof, such as in antibodies that contain a Fc region (e.g., in a full-length IgG antibody including full-length IgG1, IgG2, IgG3 or IgG4) or in a fusion protein that contains a Fc region or a part of a Fc region (referred to as an “immunoglobulin (Ig) fusion protein”, “Fc fusion protein”, or “Fc fusion polypeptide”). The altered Fc regions of the invention have one or more amino acid substitutions (also referred to as a Fc variant herein) at positions disclosed herein relative to the sequence of a corresponding unaltered (wild-type or parent) Fc region, and have one or more properties that differ from a corresponding unaltered Fc region such as increased binding to one or more Fc receptors. Although a particular antibody was employed as a parent antibody into which Fc alterations were introduced, as described in more detail hereinbelow, it will be apparent to the ordinarily skilled artisan that such Fc alterations can be incorporated into essentially any antibody or Fc fusion polypeptide using standard molecular biology techniques, and all such altered antibodies and Fc fusion polypeptides are intended to be encompassed by the invention. Fc refers to the last two constant region Ig domains of IgA, IgD, and IgG, and the last three constant region Ig domains of IgE and IgM, and the flexible hinge N-terminal to these domains. For IgA and IgM, Fc may include the J chain. Fc is bound by receptors, FcRs, which are present on certain cells. As the affinity of the interaction between Fc and certain FcRs present on particular cells correlates with targeted cytotoxicity, and clinical efficacy in humans correlates with the allotype of high or low affinity polymorphic forms of certain FcRs, an antibody or fusion polypeptide with a Fc region optimized for binding to one or more FcRs may result in more effective destruction of cancer cells.

Accordingly, in certain embodiments, the altered Fc regions of the invention impart improved properties to a polypeptide or a complex which includes a polypeptide into which the Fc region is incorporated, e.g., a complex such as a full-length antibody which includes an Ig heavy chain having an altered Fc region, such as increased binding to one or more FcRs, including but not limited to CD16, CD32 and/or CD64, and/or increased antibody dependent cellular cytotoxicity (ADCC), as compared to a corresponding polypeptide or complex, such as an antibody, incorporating a corresponding unaltered (a wild-type or parent) Fc region. A corresponding polypeptide or antibody that lacks one or more of the Fc region modifications disclosed herein and differs in FcR binding as compared to a polypeptide or antibody incorporating a Fc region of the invention, may have a native (wild-type) Fc region sequence or may have a Fc region sequence with amino acid sequence modifications (such as additions, deletions and/or substitutions) other than those disclosed herein that result in increased binding to at least one FcR.

In some embodiments of the invention, the altered Fc regions of the invention have increased binding to human CD16 as compared to a corresponding unaltered Fc region, such as increased binding to human CD16-Val (the valine 158 allotype of human CD16) and/or to human CD16-Phe (the phenylalanine 158 allotype of human CD16) as compared to a corresponding unaltered Fc region. In certain embodiments, an altered Fc region of the invention has increased binding to human CD32 (e.g., human CD32b, human CD32a-histidine 131 allotype, and/or human CD32a-arginine 131 allotype) as compared to a corresponding unaltered Fc region. In some embodiments of the invention, an altered Fc region of the invention has increased binding to human CD16 but has substantially the same binding or has decreased binding to human CD32 as compared to a corresponding unaltered Fc region. In some embodiments of the invention, an altered Fc region of the invention has increased binding to human CD64 as compared to a corresponding unaltered Fc region. In some embodiments of the invention, an altered Fc region of the invention has increased binding to human CD16 but has substantially the same binding or has decreased binding to human CD64 as compared to a corresponding unaltered Fc region. In some embodiments of the invention, an altered Fc region of the invention may have either increased or decreased binding to mouse CD16 (e.g., mouse CD16-1 and/or mouse CD16-2, mouse CD16-2 is also known as FcRIV) and/or mouse CD32 as compared to a corresponding unaltered Fc region. In some embodiments of the invention, an altered Fc region of the invention has either increased or decreased binding to monkey CD16 (e.g., cynomolgus monkey CD16) as compared to a corresponding unaltered Fc region. In some embodiments of the invention, an altered Fc region of the invention imparts increased ADCC activity to an antibody containing the altered Fc region as compared to a corresponding antibody containing an unaltered Fc region. Assays to detect or determine Fc binding (specificity and/or affinity) and/or ADCC activity are well-known to the art.

In some embodiments of the invention, altered Fc regions of the invention impart the following properties, as compared to a wild-type or parent Fc region: increased binding to human CD16, with substantially the same or decreased binding to human CD32b. Additional or alternative properties for altered Fc regions with the foregoing two properties may include substantially the same or increased binding to human CD32a and/or substantially the same or increased binding to human CD64. In some embodiments of the invention, altered Fc regions of the invention impart the following properties, as compared to a wild-type or parent Fc region: increased binding to FcRn, decreased half life, decreased binding to FcRn, or increased half life. Examples of altered Fc regions having one or more of these properties are described herein.

In one embodiment of the invention, an altered Fc region of the invention contains one of the substitutions described herein. In other embodiments, an altered Fc region of the invention contains two, three, four, or more substitutions described herein in combination. In one embodiment an altered Fc region of the invention has fifteen or fewer, e.g., ten, seven, five, or three or fewer, or any integer from two to fifteen, substitutions described herein. In another embodiment, the invention includes a polypeptide having an altered Fc region of the invention, i.e., it is an Fc fusion polypeptide, that contains one of the substitutions described herein. In one embodiment, the non-Fc region of the fusion polypeptide includes a target binding molecule. In other embodiments, the invention includes a polypeptide having an altered Fc region of the invention that contains two, three, four, or more substitutions described herein in combination. In one embodiment, the invention includes an antibody or antigen-binding antibody fragment having an altered Fc region of the invention that contains one of the substitutions described herein. In other embodiments, the invention includes an antibody or antigen-binding antibody fragment having an altered Fc region of the invention that contains two, three, four, or more substitutions described herein in combination.

In yet other embodiments, an altered Fc region of the invention contains one of the substitutions described herein as well as one or more other substitutions, which other substitutions may impart properties other than those associated with the substituted position(s) and/or substitutions in the altered Fc regions of the invention, or may additively or synergistically enhance the properties of the altered Fc regions of the invention. In another embodiment, an altered Fc region of the invention contains two, three, four, or more substitutions described herein in combination, as well as one or more other substitutions, which other substitutions may impart properties other than those associated with the substituted position(s) and/or substitutions in the altered Fc regions of the invention, or may additively or synergistically enhance the properties of the altered Fc regions of the invention.

In another embodiment, the invention includes a polypeptide, e.g., one in a complex of polypeptides such as an antibody or a Fc fusion polypeptide, or a conjugate which includes a Fc region conjugated to another molecule (a Fc fusion conjugate), having an altered Fc region of the invention that contains one of the substitutions described herein as well as one or more other substitutions, which other substitutions may impart properties other than those associated with the substituted position(s) and/or substitutions in the altered Fc regions of the invention, or may additively or synergistically enhance the properties of the altered Fc regions of the invention. In other embodiments, the invention includes a polypeptide, e.g., one in a complex of polypeptides such as an antibody or a Fc fusion polypeptide, or a conjugate which includes a Fc region conjugated to another molecule, having an altered Fc region of the invention that contains two, three, four, or more substitutions described herein in combination, as well as one or more other substitutions, which other substitutions may impart properties other than those associated with the substituted position(s) and/or substitutions in the altered Fc regions of the invention, or may additively or synergistically enhance the properties of the altered Fc regions of the invention. In some embodiments of the invention, a polypeptide with an altered Fc region of the invention has one or more of the functional properties described herein.

For polypeptides with altered Fc regions of the invention that also include a non-FcR target binding molecule domain, which optionally together with other polypeptides may form an antibody, the target binding molecule domain or variable regions of the antibody may specifically bind virtually any target molecule or antigen.

Accordingly, in one aspect, the invention pertains to a polypeptide having a Fc region (e.g., an IgG Fc region, such as an IgG1 Fc region) with at least one amino acid substitution at at least one of the following amino acid residues (positions) in a Fc region: 240, 247, 254, 268, 272, 274, 290, 295, 301, 307, 308, 312, 326, 330, 334, 343, 345, 350, 351, 352, 353, 354, 356, 357, 359, 361, 362, 363, 366, 367, 369, 372, 376, 377, 378, 379, 382, 383, 385, 394, 396, 397, 399, 401, 404, 405, 408, 410, 413, 417, 418, 419, 420, 426, 427, 437, 439, 441 or 446, or substitutions at any combination of those positions, and optionally substitutions at other positions. For all positions discussed herein, numbering is according to the EU index as in Kabat (Kabat et al., 1991). Those skilled in the art of antibodies will appreciate that this convention consists of nonsequential numbering in specific regions of an Ig sequence, enabling a normalized reference to conserved positions in Ig families. Thus, the positions of any given Ig as defined by the EU index will not necessarily correspond to its sequential sequence. In some embodiments of the invention, a polypeptide having an altered Fc region has at least one amino acid substitution at at least one of the following amino acid residues in a Fc region: 274, 308, 343, 350, 351, 352, 353, 354, 357, 359, 363, 366, 367, 369, 372, 377, 385, 394, 396, 397, 399, 404, 405, 408, 410, 417, 420, 426, 427, 441 or 446, or substitutions at any combination of those positions, and optionally substitutions at other residues. In some embodiments, a polypeptide having an altered Fc region has a plurality of substitutions at one of the following amino acid residues in a Fc region: 240, 247, 254, 268, 272, 274, 290, 295, 301, 307, 308, 312, 326, 330, 334, 343, 345, 350, 351, 352, 353, 354, 356, 357, 359, 361, 362, 363, 366, 367, 369, 372, 376, 377, 378, 379, 382, 383, 385, 394, 396, 397, 399, 401, 404, 405, 408, 410, 413, 417, 418, 419, 420, 426, 427, 437, 439, 441 or 446, and optionally substitutions at other residues. Certain substitutions of the invention include the following: V240Q, P247C, P247F, P247H, P247I, P247L, P247M, P247N, P247T, P247V, S254W, H268D, H268E, E272D, K274D, K274G, K274H, K274P, K274S, K274T, K290D, K290T, Q295C, R301S, T307A, T307C, T307D, T307E, T307F, T307G, T307I, T307K, T307L, T307M, T307N, T307P, T307R, T307V, T307Y, V308P, D312L, D312T, K326C, K326I, K326L, K326T, K326V, A330F, K334I, K334P, K334T, K334V, P343A, P343R, E345W, T350H, T350K, T350W, L351R, P352W, P353R, S354K, S354R, E356K, E357R, T359R, N361W, N361Y, Q362M, V363Q, T366V, C367H, C367T, V369R, F372W, D376T, I377F, I377H, I377K, 1377Q, I377R, I377Y, A378I, A378P, A378V, V379Y, E382G, E382H, E382Q, E382S, E382T, E382W, E382Y, S383K, G385R, G385W, T394F, T394Q, T394W, T394Y, P396I, P396Y, V397D, V397L, V397M, D399L, D401W, F404W, F405K, S408F, S408M, S408Y, L410P, D413R, W417Q, Q418Y, Q419W, G420W, S426W, V427R, T437W, K439P, L441P and/or G446W.

In addition to the polypeptide, protein or other complex, e.g., a conjugate, incorporating an altered Fc region of the invention described herein, the invention also encompasses polynucleotides and expression vectors encoding an altered Fc region or polypeptides having an altered Fc region, including libraries of those polynucleotides and expression vectors, host cells into which such polynucleotides or expression vectors have been introduced, for instance, so that the host cell produces a polypeptide having the altered Fc region, libraries of host cells, and methods of making, culturing or manipulating the host cells or libraries of host cells. For instance, the invention includes culturing such host cells so that a polypeptide with an altered Fc region is produced, e.g., secreted or otherwise released from the host cell. Pharmaceutical compositions and kits which include a polypeptide, protein or other complex with an altered Fc region of the invention, and/or polynucleotides, expression vectors or host cells encoding polypeptides having such an altered Fc region, are also encompassed. Moreover, use of a polypeptide, protein or conjugate with an altered Fc region of the invention, such as in Fc receptor binding assays or to induce ADCC activity in vitro or in vivo, is also encompassed by the invention. The invention also provides a polypeptide, protein, conjugate, polynucleotide, expression vector, and/or host cell of the invention for use in medical therapy, as well as the use of a polypeptide, protein or other complex, polynucleotide, expression vector, and/or host cell of the invention for the manufacture of a medicament, e.g., useful to induce ADCC activity in vitro or in vivo.

### BRIEF DESCRIPTION OF THE FIGURES

FIG. 1 depicts the sequence of the anti-CD30 antibody (5F11) that was mutated to produce altered Fc regions of the invention.

FIG. 2 depicts results of binding assays.

FIG. 3A depicts ADCC activity for antibodies with an altered Fc region having a single substitution.

FIGS. 3B-C summarize binding data for antibodies with an altered Fc region having a single substitution.

FIG. 4 depicts the rank of antibodies with altered Fc regions of the invention in binding assays for huCD16-Phe. The antibodies have altered Fc regions having two or more substitutions selected from 10 particular substitutions (the “10 residue library”). Binding data for huCD32b, huCD32a-Arg, huCD64 and muCD32b are also shown.

FIG. 5 depicts results for binding assays with antibodies with altered Fc regions having two or more substitutions selected from 8 particular substitutions (the “8 residue library”). Binding data for CD16-Phe, muCD16 and huFcRn are shown.

FIG. 6 shows ADCC data for antibodies from the 8 residue library. FIG. 6A shows percent lysis by antibodies at 0.01 μg/mL and 0.5 μg/mL, and ranks the antibodies based on mean percent lysis at 0.5 μg/mL. FIG. 6B shows the rank of antibodies based on mean percent lysis of antibody with the altered Fc region/wild type at 0.5 μg/mL.

FIGS. 7A-C show ADCC versus CD16 (CD16-Val or CD16-Phe) binding results for selected antibodies with altered Fc regions having one, two or three substitutions.

FIG. 8A shows percent lysis and EC₅₀ data for antibodies with altered Fc regions of the invention having two or more substitutions.

FIG. 8B shows representative dose response curves for antibodies with altered Fc regions of the invention having two or more substitutions.

FIG. 9 summarizes the Fc region substitutions associated with the highest ADCC activity or lowest EC₅₀ values.

FIG. 10 depicts FcRn binding data for antibodies with altered Fc regions of the invention.

### DETAILED DESCRIPTION OF THE INVENTION

### Antibodies

An antibody, as used herein, is a protein having one or more polypeptides encoded by all or part of mammalian Ig genes, including polyclonal or monoclonal antibodies, which specifically binds to one or more FcRs, and, if one or more variable regions are present, the protein binds to an antigen, which protein is optionally glycosylated. A full-length antibody has a structure corresponding to the natural biological form of an antibody found in nature including variable and constant regions. For example, a full-length antibody may be a tetramer, generally with two identical pairs of two Ig chains, each pair having one light chain and one heavy chain. Each light chain includes immunoglobulin domains V_{L} and C_{L}, and for IgG, each heavy chain includes immunoglobulin domains V_{H} and C_{H}, where C_{H} includes Cγ1, Cγ2, and Cγ3. In humans, Ig genes include kappa (κ) and lambda (λ) light chain genetic loci and heavy chain genetic loci, which include constant region genes mu (μ), delta (δ), gamma (γ), sigma (σ), and alpha (α) for the IgM, IgD, IgG, IgE, and IgA isotypes, respectively. An “antibody” as used herein, unless otherwise specified, includes full-length antibodies and fragments thereof, including naturally occurring antibodies, chimeric antibodies, recombinant antibodies including humanized antibodies, or antibodies subjected to other in vitro alterations, and antigen binding fragments thereof. Chimeric antibodies are molecules in which a portion of the heavy and/or light chain is derived from a particular species or belongs to a particular antibody class or subclass, while the remainder of the chain(s) is derived from another species or belongs to another antibody class or subclass. With regard to antibody fragments, those fragments include, but are not limited to, Fab, Fab′, F(ab′)2, or other antigen-binding subsequences of antibodies, such as, single chain antibodies (Fv for example), and the like, as well as Fcs, which can be prepared by in vitro treatments of full-length antibodies or by recombinant means. Methods of preparation and purification of antibodies are known in the art (see Harlow and Lane, 1988). A polypeptide, or a protein such as an antibody or fragment thereof incorporating an altered Fc region of the invention is one which specifically binds at least one FcR. “Specifically binds” includes a binding constant in the range of at least 10⁻³ to 10⁻⁶M⁻¹, and optionally in a range of 10⁻⁷ to 10⁻¹⁰ M⁻¹, as measured by methods well known to the art.

Humanized antibodies are chimeric molecules of Igs, Ig chains or fragments thereof from two or more sources, one of which is a human source, which are further altered in primary sequence to reduce non-human Ig sequences and/or to increase sequences corresponding to those found in human antibodies, e.g., human Ig consensus sequences. Humanized antibodies include residues that form a complementary determining region (CDR) in the Fv region that are from a CDR of a non-human species such as mouse, rat or rabbit having desired properties, e.g., specificity and/or affinity for a particular antigen. In general, a humanized antibody includes substantially all of at least one, and typically two, variable domains, in which all or substantially all of the sequences in the CDR regions correspond to ‘those of non-human Ig sequences and all or substantially all of the framework regions correspond to human Ig sequences, such as human Ig consensus sequences. Replacement of non-human residues to a corresponding human residue, human residues to a corresponding consensus residue, non-human residues to a corresponding consensus residue, or human residues to a corresponding non-human residue, are based on comparisons of human Ig sequences or comparisons of human Ig sequences with non-human Ig sequences, such as rat, mouse and monkey Ig sequences, using conserved residues between species for alignment but allowing for insertions and/or deletions. Methods for humanizing non-human or chimeric antibodies and aligning antibody sequences are well known in the art.

A human antibody is an antibody obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al. (1994), Lonberg et al. (1994), and Taylor et al. (1994). A fully human antibody also can be constructed by genetic or chromosomal transfection methods, as well as phage display technology, all of which are known in the art. (See, e.g., McCafferty et al. (1990) for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats. For a review, see, e.g. Johnson and Chiswell (1993). Human antibodies may also be generated by in vitro activated B cells. (See, U.S. Pat. Nos. 5,567,610 and 5,229,275).

### Fc Sources and Fc Receptor Binding

The source of a parent Fc into which one or more substitutions are introduced to yield Fc variants of the present invention may be from any antibody class (isotype), any organism, including but not limited to humans, mice, rats, rabbits, and monkeys, and preferably mammals and most preferably humans and mice, or any source, e.g., a previously engineered antibody, e.g., a chimeric antibody or a recombinant antibody including variants modified in vitro, or selected in vitro or in vivo. Thus, the source of a parent Fc is not necessarily naturally occurring, e.g., it may be a Fc chimera, or may have one or more substitutions, insertions and/or deletions, as compared to a naturally occurring Fc region of an IgA, IgD, IgE, IgG or IgM class of antibody. Alternatively, the source of a parent Fc is a Fc region from a naturally occurring antibody, including IgG1, IgG2, IgG3, IgG4, IgA1, or IgA2.

A parent Fc region to be modified may be selected for its FcR binding affinity and/or FcR binding pattern, and an altered Fc region of the invention has at least an enhanced affinity for at least one FcR, but may otherwise have the same pattern of FcR binding, as the parent Fc region.

A parent Fc region is preferably one that interacts with one or more FcRs, including but not limited to FcγRs, FcαRs, Fc∈Rs, FcμRs, FcδRs, FcRn, and viral FcγR. An altered Fc region of the invention derived from such a parent Fc region is one that has an enhanced interaction with one or more FcRs and enhanced ADCC, relative to the parent Fc region. In another embodiment, a parent Fc region does not interact with one or more FcRs, and the introduction of one or more substituted positions and/or substitutions of the invention to the parent Fc region sequence yields an altered Fc region of the invention that has enhanced interaction (affinity) with one or more FcRs and has enhanced ADCC, relative to the parent Fc region. In one embodiment, a parent Fc region elicits ADCC, and the introduction of one or more substituted positions and/or substitutions of the invention, yields an altered Fc region with enhanced ADCC relative to parent Fc region. ADCC generally requires the Fc region to be combined with a binding domain (e.g., an antibody variable domain). Methods to detect FcR binding and ADCC are known to the art.

FcRs are defined by their specificity for immunoglobulin isotypes. For example, FcRs for IgG antibodies are referred to as FcγR, those for IgE as Fc∈R, and those for IgA as FcαR. Another type of FcR is the neonatal FcR (FcRn). FcRn is structurally similar to the major histocompatibility complex (MHC) and consists of an α-chain noncovalently bound to β2-microglobulin. In humans, the FcRs for the IgG class include FcγR1 (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2). Mouse FcγRs include but are not limited to FcγRI (CD64), FcγRII (CD32), FcγRIII (CD16), and FcγRIV (CD16-2). FcγRI, FcγRIIa/c, and FcγRIIIa are positive regulators of immune complex-triggered activation, characterized by having an intracellular domain that has an immunoreceptor tyrosine-based activation motif (ITAM), while FcγRIIb has an immunoreceptor tyrosine-based inhibition motif (ITIM) and is therefore inhibitory.

FcRs are expressed in a variety of immune cells including monocytes, macrophages, neutrophils, dendritic cells, eosinophils, mast cells, platelets, B cells, large granular lymphocytes, Langerhans' cells, natural killer (NK) cells, and γγ T cells. Formation of the Fc/FcγR complex recruits these effector cells to sites of bound antigen, typically resulting in signaling events within the cells and subsequent immune responses such as release of inflammation mediators, B cell activation, endocytosis, phagocytosis, and cytotoxic attack. The cell-mediated reaction where nonspecific cytotoxic cells that express FcγRs recognize bound antibody on a target cell and subsequently cause lysis of the target cell is referred to as ADCC. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood or PBMCs, including cells cultured from blood or fractions thereof, or may be permanent cell lines.

All FcγRs bind the same region on IgG Fc, at the N-terminal end of the Cγ2 domain and the preceding hinge. In particular, the binding site on IgG for FcγR likely includes residues in the lower hinge region, i.e., residues 233-239 (EU index numbering as in Kabat et al., supra), although other regions may be involved in binding, e.g., G316-K338 (human IgG for human FcγRI), K274-R301 (human IgG1 for human FcγRIII), Y407-R416 (human IgG for human FcγRIII), as well as N297 and E318 (murine IgG2b for murine FcγRII). FcRs may bind Fc regions of the same isotype with different activities. For instance, IgG1 and IgG3 typically bind substantially better to FcγRs than IgG2 and IgG4. FcRs also differ in expression pattern and levels on different immune cells. For example, in humans, FcγRIIIB is found only on neutrophils, whereas FcγRIIIA is found on macrophages, monocytes, natural killer (NK) cells, and a subpopulation of T-cells. FcγRIIIA is the only FcR present on NK cells, one of the cell types implicated in ADCC. Moreover, there are a number of FcγR polymorphisms, some of which are associated with higher binding affinities. Further, efficient Fc binding to FcγR is associated with N-linked glycosylation at position 297, and alterations in the composition of the N297 carbohydrate or its elimination affects FcR binding. Glycosylation at other sites may affect FcR binding as well. As discussed herein, an altered Fc region of the invention, or polypeptides or protein containing complexes which include that altered Fc region, may include modifications that alter the glycosylation of the Fc region and/or other regions of the polypeptide or protein.

With respect to the neonatal receptor FcRn, a site on Fc between the Cγ2 and Cγ3 domains mediates the recycling of endocytosed antibody from the endosome back to the bloodstream, and the binding to proteins A and G.

### C1q Binding

In the same way that Fc/FcγR binding mediates ADCC, Fc/C1q binding mediates CDC. C1q forms a complex with the serine proteases C1r and C1s to form the C1 complex, the first component of the CDC pathway. C1q is capable of binding six antibodies, although binding to two IgGs or one IgM is sufficient to activate the complement cascade. Similar to the Fc interaction with FcγRs, different IgG subclasses have different affinity for C1q.

The Fc region which may be involved in complement fixation includes amino acid residues 318 to 337. There may be at least two different regions involved in the binding of C1q: one on the β-strand of the CH2 domain bearing the Glu318, Lys320 and Lys322 residues, and the other on a turn located in close proximity to the same β-strand, and containing a particular residue at position 331. However, other residues such as residues Leu235 and Gly237 located in the lower hinge region of human IgG1, may play a role in complement fixation and activation, i.e., the amino acid residues necessary for C1q and FcR binding of human IgG1 may be located in the N-terminal region of the CH2 domain, i.e., residues 231 to 238. The ability of IgG to bind C1q and activate the complement cascade may also depend on the presence, absence, or modification of the carbohydrate moiety positioned between the two CH2 domains in Fc (which is normally anchored at Asn297).

### Fc Containing Fusions

A Fc containing fusion includes a polypeptide where a Fc region with favorable FcR binding, and optionally favorable pharmacokinetics, is linked to one or more molecules. The linkage may be synthetic in nature, e.g., via chemical conjugation, or via recombinant expression, i.e., a fusion polypeptide is formed. Thus, the molecule linked to a Fc region may be a molecule useful to isolate or purify the Fc region, e.g., a tag such as a Flag-tag, Strep-tag, glutathione S transferase, maltose binding protein (MBP) or a His-tag, or other heterologous polypeptide, e.g., a ligand for a receptor, an extracellular domain of a receptor, or a variable region of a heavy Ig chain, and/or another molecule. A heterologous polypeptide is a polypeptide that is not naturally (in nature) associated with a particular Fc region and optionally binds a target molecule. For instance, the heterologous polypeptide may be an enzyme, a receptor, e.g., an extracellular domain of a receptor, or other protein or protein domain that binds another (target) molecule. The heterologous polypeptide of the fusion may correspond to a full-length (wild-type) polypeptide or a target-binding fragment thereof. A heterologous polypeptide may have a sequence that differs from that of a corresponding native (wild-type) or parent polypeptide sequence by virtue of at least one amino acid substitution, e.g., from about one to about twenty amino acid substitutions, i.e., it is a variant heterologous polypeptide, but has substantially the same activity, e.g., substantially the same target binding activity, as the corresponding native or parent polypeptide. A variant polypeptide sequence has at least about 80% homology with a wild-type or parent polypeptide sequence, and most preferably at least about 90% homology, more preferably at least about 95% homology, with a wild-type or parent polypeptide sequence.

### Exemplary Methods to Prepare Polynucleotides of the Invention

An “isolated” polynucleotide is a nucleic acid molecule that is separated from at least one contaminant polynucleotide, polypeptide and/or other molecule with which it is ordinarily associated in a cell or cell-free composition. Thus, an isolated polynucleotide is in a form or setting different than which it is found in cells or a cell-free composition containing that polynucleotide. In one embodiment, an isolated polynucleotide may form part of a linear or circular vector, such as an expression vector where the polynucleotide is linked to transcription and/or translation control sequences or other sequences.

Methods known to the art may be employed to prepare polynucleotides or a library of polynucleotides encoding an altered Fc region or a polypeptide with an altered Fc region (a Fc fusion polypeptide), from one or more polynucleotides encoding a wild-type or parent Fc region or a polypeptide with a wild-type or parent Fc region, and optionally isolate a particular polynucleotide. Methods to prepare a polynucleotide or a library of polynucleotides encoding an altered Fc region (or a fragment thereof which can be introduced into a Fc region or Fc region containing polypeptide by PCR, ligation, recombination or other techniques) or a polypeptide with an altered Fc region include, but are not limited to, site-directed (or oligonucleotide-mediated) mutagenesis, saturation mutagenesis, PCR mutagenesis, or cassette mutagenesis of a wild-type or parent polynucleotide having an open reading frame to be modified.

Site-directed mutagenesis is well known in the art (see, e.g., Carter et al., 1985 and Kunkel et al., 1987). Briefly, in carrying out site-directed mutagenesis of DNA, the starting DNA is altered by first hybridizing at least one oligonucleotide encoding a desired mutation(s) to a single strand of starting wild-type or parent DNA. After hybridization, a DNA polymerase is used to synthesize an entire second strand, using the hybridized oligonucleotide(s) as a primer, and using the single strand of the starting DNA as a template. Thus, the oligonucleotide(s) encoding the desired mutation(s) is incorporated in the resulting double-stranded DNA.

PCR mutagenesis is also suitable for making polynucleotides encoding a polypeptide with one or more amino acid substitutions relative to a wild-type or parent polypeptide. See Higuchi, 1990 and Vallette et al., 1989). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., 1985. The starting material is a plasmid (or other vector) with the wild-type or parent DNA to be mutated. The codon(s) in the starting DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the wild-type or parent DNA. The plasmid DNA is cut at these sites to linearize it. A double-stranded oligonucleotide having the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Yet another method to prepare polynucleotides encoding variant polypeptides, e.g., in a Fc region or non-Fc sequences, is saturation mutagenesis. Codon primers containing a degenerate N,N,G/T sequence are used to introduce point mutations into a polynucleotide, so as to generate a set of progeny polypeptides in which a full range of single amino acid substitutions is represented at each amino acid position, see, e.g., U.S. Pat. Nos. 6,171,820, 6,562,594 and 6,764,835, the disclosures of which are incorporated by reference herein. These oligonucleotides can include a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (e.g., one degenerate N,N,G/T cassette, is used for subjecting each original codon in a parental polynucleotide template to a full range of codon substitutions. In another aspect, at least two degenerate cassettes are used, either in the same oligonucleotide or not, for subjecting at least two original codons in a parental polynucleotide template to a full range of codon substitutions. For example, more than one N,N,G/T sequence can be contained in one oligonucleotide to introduce amino acid substitutions at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

For example, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g., in an oligonucleotide) a degenerate triplet sequence having only one N, where said N can be in the first second or third position of the triplet. Any other bases including any combinations and permutations thereof can be used in the remaining two positions of the triplet. Alternatively, it may be desirable in some instances to use a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host using an expression vector). The progeny polypeptides are then subjected to screening for one or more properties. For instance, the altered Fc regions described herein were prepared by saturation mutagenesis and identified by screening for binding to one or more FcRs, as described below. When an individual progeny polypeptide is identified by screening to display a favorable change in property, it can be sequenced to identify the correspondingly favorable amino acid substitution contained therein.

In one aspect, upon mutagenizing each and every amino acid position in a parental polypeptide using saturation mutagenesis, favorable amino acid changes may be identified at more than one amino acid position. One or more new progeny molecules can be generated that contain a combination of all or part of these favorable amino acid substitutions. For instance, site-saturation mutagenesis can be used together with another stochastic or non-stochastic means, e.g., in an interactive manner, to vary sequence, e.g., synthetic ligation reassembly (SLR), shuffling, chimerization, recombination and other mutagenizing processes and mutagenizing agents. Methods useful to prepare nucleic acids encoding variant antigen binding sites, e.g., in antibodies, are disclosed, for instance, in U.S. published application 20030219752.

SLR is a directed evolution process to generate variant polypeptides which employs ligating oligonucleotide fragments together non-stochastically. The method differs from stochastic oligonucleotide shuffling in that the nucleic acid building blocks are not shuffled, concatenated or chimerized randomly, but rather are assembled non-stochastically. See, e.g., U.S. Pat. Nos. 6,537,776 and 6,605,449. In one aspect, SLR includes: (a) providing a template polynucleotide that includes a sequence for a homologous gene; (b) providing a plurality of building block polynucleotides, which are designed to cross-over reassemble with the template polynucleotide at a predetermined sequence, and where a building block polynucleotide includes a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides having homologous gene sequence variations.

SLR does not depend on the presence of high levels of homology between polynucleotides to be rearranged. Thus, the method can be used to non-stochastically generate libraries (or sets) of progeny molecules with over 10¹⁰⁰ different chimeras, and over 10¹⁰⁰⁰ different progeny chimeras. Thus, polynucleotides encoding Fc region containing polypeptides of the invention may be prepared by non-stochastic methods by producing a set of finalized chimeric polynucleotides encoding a Fc region containing polypeptide having an overall assembly order that is chosen by design. This method includes the steps of generating, by design, a plurality of specific nucleic acid building blocks having serviceable mutually compatible ligatable ends, and assembling these nucleic acid building blocks, such that a designed overall assembly order is achieved. The mutually compatible ligatable ends of the nucleic acid building blocks to be assembled are considered to be “serviceable” for this type of ordered assembly if they enable the building blocks to be coupled in predetermined orders. Accordingly, the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g., T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotides. These parental oligonucleotide templates serve as a source of sequence information that aids in the design of the nucleic acid building blocks that are to be mutagenized, e.g., chimerized or shuffled. In one aspect of this method, the sequences of a plurality of parental nucleic acid templates are aligned in order to select one or more demarcation points. The demarcation points can be located at an area of homology, and include one or more nucleotides. These demarcation points are preferably shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (having at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more preferably a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

A ligation reassembly process may be performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another aspect, the assembly order (i.e., the order of assembly of each building block in the 5′ to 3′ sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g., one by one. In other words, through the selective use of specific nucleic acid building blocks, coupled with the selective use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups.

Because of its ability to perform chimerizations in a manner that is highly flexible yet exhaustive and systematic as well, particularly when there is a low level of homology among the progenitor molecules, these methods provide for the generation of a library (or set) of a large number of progeny molecules. Because of the non-stochastic nature of the instant ligation reassembly invention, the progeny molecules generated preferably include a library of finalized chimeric nucleic acid molecules having an overall assembly order that is chosen by design. The saturation mutagenesis and optimized directed evolution methods also can be used to generate different progeny molecular species.

It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the method. In fact, demarcation points can even be chosen in areas of little or no intermolecular homology. For example, because of codon wobble, i.e., the degeneracy of codons, nucleotide substitutions can be introduced into nucleic acid building blocks without altering the amino acid originally encoded in the corresponding progenitor template. Alternatively, a codon can be altered such that the coding for an original amino acid is altered. This invention provides that such substitutions can be introduced into the nucleic acid building block in order to increase the incidence of intermolecular homologous demarcation points and thus allows for an increased number of couplings to be achieved among the building blocks, which in turn allows a greater number of progeny chimeric molecules to be generated.

The synthetic nature of the step in which the building blocks are generated allows the design and introduction of nucleotides (e.g., one or more nucleotides, which may be, for example, codons or introns or regulatory sequences) that can later be optionally removed in an in vitro process (e.g., by mutagenesis) or in an in vivo process (e.g., by utilizing the gene splicing ability of a host organism). It is appreciated that in many instances the introduction of these nucleotides may also be desirable for many other reasons in addition to the potential benefit of creating a serviceable demarcation point.

In one aspect, a nucleic acid building block is used to introduce an intron. Thus, functional introns are introduced into a man-made gene manufactured according to the methods described herein. The artificially introduced intron(s) can be functional in a host cells for gene splicing much in the way that naturally-occurring introns serve functionally in gene splicing.

Methods employed to prepare a polynucleotide or libraries of polynucleotides encoding altered Fc regions may also be employed to introduce other modifications to a Fc region or a Fc region containing polypeptide, modifications including but not limited to substitution, insertion and/or deletion of amino acid residues, prior to, concurrently, or after polynucleotides with altered Fc regions are prepared. The other introduced substitutions may result in altered FcR binding and/or ADCC activity, but the introduction of those other substitutions preferably does not substantially decrease FcR binding activity and/or ADCC activity altered by introduction of one or more substitutions at positions described herein which yield an altered Fc region of the invention, and/or may alter one or more other desirable activities, e.g., substitution(s) introduced into a non-Fc region of a fusion polypeptide may enhance binding to a target molecule other than a FcR.

For instance, a Fc region alteration that modifies FcR binding may be combined with substitution of a cysteine not involved in maintaining the proper conformation of the resulting polypeptide, generally with serine, which may improve stability and prevent aberrant cross linking, substitution to alter the glycosylation pattern of the resulting polypeptide may improve stability or function of the resulting polypeptide and/or substitution to alter the class, subclass or allotype of the Fc region may alter Fc binding to particular Fc ligands. Glycosylation of polypeptides is typically either N-linked or O-linked. N-linked refers to the attachment of the carbohydrate moiety to the side chain of an asparagine residue in a sequence such as asparagine-X-serine and asparagine-X-threonine (which creates a potential glycosylation site), where X is any amino acid except praline. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to a polypeptide may be accomplished by altering the amino acid sequence such that it contains one or more of the above-described tripeptide sequences for N-linked glycosylation sites or the addition of, or substitution by, one or more serine or threonine residues to the sequence of the original polypeptide for O-linked glycosylation.

### Vectors and Host Cells

Vectors useful in the invention include nucleic acid sequences encoding at least a portion of a Fc region, e.g., a region that includes a portion of Fc residues 240 to 446, or a portion of a Fc ligand, e.g., the extracellular domain of a FcR. In one embodiment, the vector encodes an altered Fc region of the invention or a polypeptide incorporating a Fc region. Other sequences that may be included in vectors include a targeting peptide, e.g., a signal peptide from an Ig gene or a non-Ig gene, a tag useful to isolate or purify the encoded polypeptide, e.g., a GST or a His tag, an origin of replication, a selectable marker or reporter gene, a promoter, an enhancer, a polyA addition site, splice sites, introns, and/or other control sequences. Vectors may be circular, e.g., a plasmid, or linear, e.g., a cosmid. Certain vector sequences, e.g., promoters, origins of replication and/or selectable markers, may only be employed with particular host cells, e.g., prokaryotic cells, such as E. coli, Streptomyces, Pseudomonas and Bacillus, or eukaryotic cells, such as yeast, e.g., Picchia, Saccharomyces or Schizosaccharomyces, insect cells, avian cells, plant cells, or mammalian cells, e.g., human, simian, parcine, ovine, rodent, bovine, equine, caprine, canine or feline cells

Control sequences are DNA sequences for the expression of an operably linked open reading frame, e.g., for an altered Fc region, in a particular host organism. Control sequences suitable for prokaryotes, for example, include but are not limited to a promoter, an operator sequence, and/or a ribosome binding site. Control sequences for eukaryotic cells include but are not limited to promoters, polyA addition sites and/or enhancers. Promoters may be regulatable, e.g., inducible, or constitutive. The selection of a particular promoter, and optionally enhancer, depends on what cell type is to be used for expression. Some eukaryotic promoters and enhancers have a broad host range while others are functional in a limited subset of cell types.

A particular nucleic acid is operably linked to another nucleic acid when they are placed in a functional relationship with one another. For example, DNA for a peptide tag or secretory leader sequence is operably linked to an open reading frame for a particular polypeptide if, generally the sequences are in the same reading frame, and the expression of operably linked sequences yield a fusion protein containing sequences for the tag or secretory leader sequence and the particular polypeptide; a promoter or enhancer is operably linked to an open reading frame if it affects the transcription of the open reading frame; or a ribosome binding site is operably linked to an open reading frame if it is positioned so as to facilitate translation. Some transcription control sequences such as enhancers do not have to be contiguous with (in close proximity to) an open reading frame to alter transcription of that open reading frame. Linking of sequences may be accomplished by ligation at convenient restriction sites or by employing the synthetic adaptors or linkers in accordance with conventional practice.

An origin replication (or autonomously replicating sequences) enables the vector to replicate in one or more selected host cells, generally, independently of the host chromosomal DNA. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, EBV, VSV or BPV) are useful for cloning vectors in mammalian cells.

A selectable marker gene or a reporter gene, or both, may be included in a vector to facilitate identification and selection of transformed cells from the population of cells sought to be transformed. Alternatively, the selectable marker or reporter gene may be carried on a separate piece of DNA and used in a co-transformation procedure. Both selectable marker and reporter genes may be flanked with appropriate control sequences to enable expression in the host cells. A selectable marker gene typically encodes a protein that confers resistance to antibiotics or other toxins, e.g., ampicillin, neomycin, methotrexate, or tetracycline, complements auxotrophic deficiencies, or supplies critical nutrients not available from complex media. Examples of dominant selection employ the drugs neomycin, mycophenolic acid and hygromycin. Another example of suitable selectable marker genes for mammalian cells allow for genes encoding DHFR, thymidine kinase, metallothionein-I and -II, adenosine deaminase, ornithine decarboxylase, and the like.

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Preferred genes include the chloramphenicol acetyl transferase gene (cat) from Tn9 of E. coli, the beta-glucuronidase gene (gus) of the uidA locus of E. coli, and the luciferase gene from firefly Photinus pyralis.

Expression vectors usually include a promoter that is recognized by the host organism and is operably linked to a polynucleotide encoding a polypeptide. Promoters suitable for use with prokaryotic hosts include but are not limited to the phoA promoter, β-lactamase and lactose promoter systems, alkaline phosphatase, a tryptophan (trp) promoter system, ahybrid promoters such as the tac promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Promoters for use in bacterial systems also will contain a Shine-Dalgarno sequence operably linked to the DNA encoding the polypeptide.

Many, if not all, eukaryotic promoter sequences have an AT-rich region located approximately 25 to 30 bases upstream from the site where transcription is initiated, and another sequence found 70 to 80 bases upstream from the start of transcription of many genes is a CNCAAT region where N may be any nucleotide. Thus, any naturally occurring or synthetic eukaryotic promoter with these sequences may be employed in eukaryotic expression vectors. Transcription from vectors in mammalian host cells may be controlled, for example, by promoters such as promoters from polyoma virus, fowlpox virus, adenovirus (such as Adenovirus 2), bovine papilloma virus, avian sarcoma virus, cytomegalovirus, a retrovirus, hepatitis-B virus, HSV thymidine kinase promoter and Simian Virus 40 (SV40), or from heterologous mammalian promoters, e.g., the actin promoter, metallothionein-I promoter or heat-shock promoters, provided such promoters are compatible with the host cell systems. Other promoters known to control expression of genes in prokaryotic or eukaryotic cells or their viruses may also be used.

For expression in yeast, promoters for 3-phosphoglycerate kinase or other glycolytic enzymes, such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, or glucokinase may be employed. Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Yeast enhancers also are advantageously used with yeast promoters.

Transcription of a polynucleotide encoding a polypeptide may be increased by inserting an enhancer sequence into the vector either 5′ or 3′ to the open reading frame. Many enhancer sequences are now known from mammalian genes (globin, elastase, albumin, α-fetoprotein, and insulin), and viruses, e.g., the SV40 enhancer, the CMV early promoter enhancer, the polyoma enhancer, and adenovirus enhancers.

Expression vectors used in eukaryotic host cells (yeast, fungi, insect, plant, animal, human, or nucleated cells from other multicellular organisms) preferably also contain sequences necessary for the termination of transcription and for stabilizing the mRNA. At the 3′ end of most eukaryotic genes is an AATAAA sequence that may be the signal for addition of the poly A tail to the 3′ end of the coding sequence. Efficient expression of recombinant DNA sequences in eukaryotic cells requires expression of signals directing the efficient termination and polyadenylation of the resulting transcript. The term “poly A site”, “polyA addition site” or “poly A sequence” denotes a DNA sequence which directs both the termination and polyadenylation of the nascent RNA transcript. Such sequences are commonly available from the 5′ and, occasionally 3′, untranslated regions of eukaryotic or viral DNAs or cDNAs, or may be synthetic in nature.

Host cells augmented with vector sequences are typically produced by transfection with a DNA sequence in a plasmid expression vector, a viral expression vector, or as an isolated linear DNA sequence. An isolated polynucleotide of interest can be readily introduced into the host cells, e.g., plant, mammalian, bacterial, yeast or insect cells, by transfection with an expression vector having the polynucleotide, by any procedure useful for the introduction into a particular cell, e.g., physical or biological methods, to yield a transformed cell having the polynucleotide stably integrated into its genome, or stably maintained extrachromosomally, which polynucleotide is expressed by the host cell.

Physical methods to introduce a vector into a host cell include calcium phosphate precipitation, lipofection, particle bombardment, microinjection, electroporation, and the like. Biological methods to introduce the vector into a host cell include the use of DNA and RNA viral vectors. The main advantage of physical methods is that they are not associated with pathological or oncogenic processes of viruses. However, they are less precise, often resulting in multiple copy insertions, random integration, disruption of foreign and endogenous gene sequences, and unpredictable expression. For mammalian gene therapy, it is desirable to use an efficient means of precisely inserting a single copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like. For plant cells, a vector may be introduced to plant protoplasts using bombardment techniques or to cells via biological means, e.g., Agrobacterium or plant virus-mediated methods.

Once a vector encoding, for example, a Fc region such as an altered Fc region of the invention or Fc region containing polypeptide such as an Ig heavy chain with an altered Fc region or other Fc fusion polypeptide, the vector may be introduced into a host cell, optionally along with other vectors, e.g., a vector encoding an Ig light chain, or into a host cell modified to express another polypeptide such as an Ig light chain, or into an in vitro transcription/transcription reaction, so as to express the encoded polypeptide. For some expression systems, host cells may be cultured in conventional nutrient media modified as appropriate for inducing promoters, selecting transformants, or amplifying desired sequences. A resulting polypeptide with an altered Fc region is optionally isolated, e.g., from host cell supernatants, and screened for one or more activities. In one embodiment, the Fc region may be one that is anchored to the surface of a cell, e.g., via fusion with a transmembrane domain.

Suitable host cells for expressing the polynucleotide in the vectors are the prokaryotic, yeast, or higher eukaryotic cells. Suitable prokaryotes for this purpose include eubacteria, such as Gram-negative or Gram-positive organisms, for example, Enterobacteriaceae such as Escherichia, e.g., E. coli, Enterobacter, Erwinia, Kiebsiella, Proteus, Salmonella, e.g., Salmonella typhimurium, Serratia, e.g., Serratia marcescans, and Shigella, as well as Bacilli such as B. subtilis, Pseudomonas such as P. aeruginosa, and Streptomyces.

Eukaryotic microbes such as filamentous fungi or yeast are also suitable cloning or expression hosts for polypeptide variant-encoding vectors. Saccharomyces cerevisiae, Schizosaccharomyces pombe, Kluyveromyces hosts such as, e.g., K. lactis, K. fragilis, K. bulgaricus, K. wickeramii, K. waltii, K. drosophilarum, K. thermotolerans, and K. marxianus; Pichia pastoris, Candida, Trichoderma reesia, Schwanniomyces such as Schwanniomyces occidentalis; and filamentous fungi such as Neurospora, Penicillium, Tolypocladium, and Aspergillus hosts may be employed.

Suitable host cells for the expression of glycosylated polypeptides are derived from multicellular organisms. Examples of invertebrate cells for expression of glycosylated polypeptide include plant and insect cells. Numerous baculoviral strains and variants and corresponding permissive insect host cells from hosts such as Spodoptera frugiperda, Aedes aegypti, Aedes albopictus, Drosophila melanogaster, and Bombyx mori may be used. For instance, viral vectors may be used to introduce a polynucleotide of the invention, particularly for transfection of Spodoptera frugiperda cells. Plant cell cultures of cotton, corn, potato, soybean, petunia, tomato, and tobacco can also be utilized as hosts.

Examples of useful vertebrate cells include mammalian cells, e.g., human, simian, canine, feline, bovine, equine, caprine, ovine, swine, or rodent, e.g., rabbit, rat, mink or mouse cells.

Transgenic plants and animals may be employed as expression systems, although glycosylation patterns in those cells may be different from human glycoproteins. In one embodiment, transgenic rodents are employed as expression systems. Bacterial expression may also be employed. Although bacterially expressed proteins lack glycosylation, other alterations may compensate for any reduced activity such as poor stability and solubility, which may result from prokaryotic expression.

Optionally, a Fc region or Fc containing polypeptide is isolated from host cells, e.g., from host cell supernatants, or an in vitro transcription/translation mixture, yielding a composition. An isolated polypeptide in the composition is one which has been isolated from at least one other molecule found in host cells, host cell supernatants or the transcription/translation mixture, e.g., by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography. For some applications, the isolated polypeptide in the composition is the predominant species present (i.e., on a molar basis it is more abundant than any other individual species in the composition), and preferably comprises at least about 50 percent (on a molar basis), more preferably more than about 85%, about 90%, about 95%, and about 99, of all macromolecular species present.

The isolated Fc region or Fc containing polypeptide may be subjected to further in vitro alterations, e.g., treated with enzymes or chemicals such as proteases, molecules such as those which alter glycosylation or ones that are useful to conjugate (couple) the isolated Fc region or Fc region containing polypeptide to another molecule such as a label including but not limited to fluorescent labels (e.g., FITC, rhodamine, lanthanide, phosphors), enzymatic labels (e.g., horseradish peroxidase, β-galactosidase, luciferase, alkaline phosphatase), chemiluminescent labels, biotinyl groups, avidin groups, or polypeptide epitopes recognized by a secondary reporter (e.g., leucine zipper pair sequences, binding sites for secondary antibodies, metal binding domains, epitope tags), sugars, lipids, fats, paramagnetic molecules or sound wave emitters, metals, or synthetic polymers.

Identifying Fc Variants or Fc Variant Containing Polypeptide with Desirable

### Activities

Methods to screen for various activities associated with a Fc region as well as activities associated with polypeptides or complexes that incorporate a Fc region, activities including but not limited to FcR binding (see U.S. Pat. No. 6,737,056 and U.S. published application Serial No. 2004013210), are well known to the art. For instance, to assess ADCC activity of a Fc containing polypeptide, an in vitro and/or in vivo ADCC assay, may be performed using varying effector:target ratios, e.g., PBMC and NK cells or in a animal model, respectively.

In one embodiment, Fc containing polypeptides expressed by host cells are screened for enhanced FcRIII receptor binding affinity or activity in vitro and/or in vivo and/or ADCC activity in vitro and/or in vivo. In one embodiment, the binding of a FcRIII by a Fc containing polypeptide with an altered Fc region is at least 1.5 fold, e.g., at least 3-fold, greater than the binding of that receptor by a corresponding polypeptide with an unaltered Fc region. Thus, by introducing amino acid sequence modifications described herein in a wild-type or parent Fc region or a Fc region containing polypeptide, which wild-type or parent Fc region preferably elicits ADCC and optionally is a human Fc region, e.g., a native sequence human Fc region human IgG sequence, a variant Fc region is obtained which binds FcRIII with better affinity and mediates ADCC in the presence of human effector cells more effectively than the wild-type or parent Fc region or Fc region containing polypeptide. Moreover, altered Fc regions may be screened for differential binding to particular FcRs, as described above. For instance, soluble FcRs such as recombinant soluble human CD16 and recombinant soluble human CD32 are contacted with one or more different altered Fc regions in parallel, and altered Fc regions having one or more substitutions that enhance binding to human CD16 but not to human CD32, relative to an unaltered Fc region, are identified. Those substitutions may be combined with other substitutions that enhance binding to FcRIII, to yield a progeny Fc region, and the activities of that progeny Fc region relative to an unaltered or parent Fc region, determined. A combination of substitutions in a Fc region or Fc region containing polypeptide may yield a combinatorially altered Fc region or a combinatorially altered Fc region containing polypeptide with synergistically enhanced properties.

Other methods to identify polypeptides with altered Fc regions, including antibodies with an altered Fc region, with desirable properties, and thus a corresponding polynucleotide sequence, which method may be employed alone or in combination with methods described above, include using modeling, e.g., 3D-modeling, of altered Fc regions, preferably in the context of the molecule to be screened for activity, e.g., an antibody with the Fc region, to select for Fc regions with particular characteristics. Characteristics that may be screened for by modeling include, but are not limited to, a particular angle near FcR binding sites, hinge architecture, intra- and inter-molecular chain interactions, e.g., substitutions that promote or disrupt hydrophobic interactions or stabilize conformation in a particular region. Thus, a 3D model of a Fc region containing polypeptide having at least one of the substituted positions of the invention in combination with one or more other substitutions may be employed to identify combinations of substitutions to be introduced into a polynucleotide for expression in host cells.

### Uses

The Fc variants of the present invention, whether or not incorporated into a heterologous polypeptide, e.g., incorporated into a Fc fusion with a ligand for a cell surface receptor, e.g., CTLR-4 ligand or heavy chain of an antibody, or conjugated to a molecule of interest, as well as polynucleotides and host cells encoding those variants, optionally in combination with one or more other agents, e.g., therapeutic or research reagents, are useful in a variety of methods, e.g., in screening methods, prophylactic methods, therapeutic methods, veterinary methods and agricultural methods. The one or more other agents include other Fc region or Fc region containing polypeptides, including those with unaltered Fc regions. In one embodiment, a Fc variant is incorporated into an antibody or other Fc fusion polypeptide and that antibody or Fc fusion polypeptide, optionally in conjunction with one or more other useful compositions, employed to target particular cells. In one embodiment, a Fc variant containing antibody or an antigen-binding fragment thereof targets and optionally kill target cells that bear the target antigen. In another embodiment, a Fc variant containing antibody or an antigen-binding fragment thereof targets and activates cells that bear the target antigen, e.g., thereby increasing expression of another antigen, such as a viral or cellular antigen.

In one embodiment, the Fc variants or polypeptides incorporating a Fc variant may be used to prevent, inhibit or treat various conditions or diseases, in humans and non-humans, including non-human mammals. For example, an antibody containing an altered Fc region of the invention may be administered to a human or non-human animal which is at risk of, e.g., prone to having a disease, prior to the onset of the disease and so prevent or inhibit one or more symptoms of that disease. A Fc region or Fc region containing polypeptide, or a conjugate thereof, may be administered after clinical manifestation of a disease in a human or non-human animal to inhibit or treat the disease. In one embodiment, a pharmaceutical composition comprising an antibody or Fc fusion polypeptide of the present invention is administered to a human or non-human animal with an autoimmune, immunological, infectious, inflammatory, neurological, or neoplastic disease, e.g., cancer. Examples of cancer which may be inhibited or treated with a Fc containing polypeptide of the invention, include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer.

Furthermore, the Fc variants of the present invention may be used to treat conditions including but not limited to congestive heart failure (CHF), vasculitis, rosecea, acne, eczema, myocarditis and other conditions of the myocardium, systemic lupus erythematosus, diabetes, spondylopathies, synovial fibroblasts, and bone marrow stroma; bone loss; Paget's disease, osteoclastoma; multiple myeloma; breast cancer; disuse osteopenia; malnutrition, periodontal disease, Gaucher's disease, Langerhans' cell histiocytosis, spinal cord injury, acute septic arthritis, osteomalacia, Cushing's syndrome, monostotic fibrous dysplasia, polyostotic fibrous dysplasia, periodontal reconstruction, and bone fractures; sarcoidosis; multiple myeloma; osteolytic bone cancers, breast cancer, lung cancer, kidney cancer and rectal cancer; bone metastasis, bone pain management, and humoral malignant hypercalcemia, ankylosing spondylitisa and other spondyloarthropathies; transplantation rejection, viral infections, fungal infections, or bacterial infections. In one embodiment, the Fc variants of the present invention may be used to treat conditions including but not limited to hematologic neoplasias and neoplastic-like conditions for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia, tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, and tumors of the vascular system (angiosarcoma and hemagiopericytoma), osteoporosis, hepatitis, HIV, AIDS, spondyloarthritis, rheumatoid arthritis, inflammatory bowel diseases (IBD), sepsis and septic shock, Crohn's Disease, psoriasis, schleraderma, graft versus host disease (GVHD), allogenic islet graft rejection, hematologic malignancies, such as multiple myeloma (MM), myelodysplastic syndrome (MDS) and acute myelogenous leukemia (AML), inflammation associated with tumors, peripheral nerve injury or demyelinating diseases.

Fc regions or Fc region containing polypeptides of the invention may be administered alone or in combination with one or more other therapeutic agents, including but not limited to cytotoxic agents, e.g., chemotherapeutic agents, cytokines, growth inhibitory agents, anti-hormonal agents, kinase inhibitors, anti-angiogenic agents, cardioprotectants, or other therapeutic agents, in amounts that are effective for the purpose intended. The skilled medical practitioner can determine empirically the appropriate dose or doses of therapeutic agents including Fc regions or Fc region containing polypeptides of the present invention that are may thus be administered concomitantly with one or more other therapeutic regimens. For example, an antibody or Fc fusion polypeptide of the present invention may be administered to a patient along with chemotherapy or other therapy, e.g., other agents such as an anti-angiogenic agent, a cytokine, radioisotope therapy, or both chemotherapy and other therapies. In one embodiment, the antibody or Fc fusion of the present invention may be administered in conjunction with one or more other antibodies or Fc fusions, which may or may not comprise a Fc variant of the present invention. In one embodiment, a Fc containing polypeptide of the present invention is administered with a chemotherapeutic agent, i.e., a chemical compound useful in the treatment of cancer. A chemotherapeutic or other cytotoxic agent may be administered as a prodrug, i.e., it is in a form of a pharmaceutically active substance that is less cytotoxic to cells compared to the drug and is capable of being converted into the drug.

Pharmaceutical compositions are also contemplated having a Fc region, a Fc fusion polypeptide, antibodies having a Fc region, or conjugates thereof, that are formulated, optionally with one or more other agents. Formulations of antibodies, Fc regions, or Fc region containing polypeptides, or conjugates, of the present invention are prepared for storage by mixing the antibodies, Fc regions, or Fc region containing polypeptides, or conjugates, having the desired degree of purity with optional pharmaceutically acceptable carriers, excipients or stabilizers (Remington's Pharmaceutical Sciences 16th edition, Osol, A. Ed., 1980), in the form of lyophilized formulations or aqueous solutions. Acceptable carriers, excipients, or stabilizers are nontoxic to recipients at the dosages and concentrations employed, and include buffers such as antioxidants; alkyl parabens; low molecular weight (less than about 10 residues) polypeptides; hydrophilic polymers; amino acids; monosaccharides; and other carbohydrates; chelating agents; fillers; binding agents; additives; coloring agents; salt-forming counter-ions; metal complexes; and/or non-ionic surfactants. Other formulations includes lipid or surfactant based formulations, microparticle or nanoparticle based formulations, including sustained release dosage formulations, which are prepared by methods known in the art.

The concentration of the Fc region, antibody or other Fc region containing polypeptide of the present invention in the formulation may vary from about 0.1 to 100 weight %. In a preferred embodiment, the concentration of the Fc region, antibody or Fc fusion polypeptide is in the range of 0.001 to 2.0 M. In order to treat a patient, an effective dose of the Fc region, or antibody or other Fc region containing polypeptide, and conjugates thereof, of the present invention may be administered. By “therapeutically effective dose” herein is meant a dose that produces the effects for which it is administered. Dosages may range from 0.01 to 100 mg/kg of body weight or greater, for example 0.1, 1, 10, or 50 mg/kg of body weight, with 1 to 30 mg/kg being preferred, although other dosages may provide beneficial results. The amount administered is selected to prevent treat a particular condition or disease.

Administration of the Fc region, or antibody or other Fc region containing polypeptide, and conjugates thereof, of the present invention may be continuous or intermittent, depending, for example, upon the recipient's physiological condition, whether the purpose of the administration is therapeutic or prophylactic, and other factors known to skilled practitioners. The administration of the Fc region, or antibody or other Fc region containing polypeptide, and conjugates thereof, of the present invention may be essentially continuous over a preselected period of time or may be in a series of spaced doses. Both local and systemic administration is contemplated.

Administration of the pharmaceutical composition comprising a Fc region, an antibody or other Fc containing polypeptide and conjugates of the present invention, preferably in the form of a sterile aqueous solution, may be done in a variety of ways, including, but not limited to, orally, subcutaneously, intravenously, intranasally, intraotically, transdennally, topically, intraperitoneally, intramuscularly, intrapulmonary, inhalable technology, vaginally, parenterally, rectally, or intraocularly. In some instances, for example for the treatment of wounds, inflammation, etc., the antibody or Fc fusion may be directly applied as a solution or spray.

The invention will now be illustrated by the following non-limiting Examples.

### Example 1

### Analyses of Single Substitution Variants in the Fc Region

### Materials and Methods

### Recombinant Fc Gamma Receptors

Secreted versions of recombinant Fc gamma receptors, including cynomolgus CD16, human CD32A-Arg131, human CD32A-His131, human CD32B, human CD64, mouse CD16-1, and mouse FcRIV, were engineered by replacing their C-terminal transmembrane domains with 6XHis sequences and using the osteonectin signal peptide (MRAWIFFLLCLAGRALA; SEQ ID NO:11) as the signal sequences. The Fc gamma receptor sequences were subcloned into pcDNA3.1(HygroR)-based vectors for recombinant expression. Linearized vectors were transfected into CHO-S cells and stable cells were selected under 500 ug/mL of hygromycin. The FcRn receptor construct was co-transfected into 293 cells with an expression construct encoding beta2 microglobulin. The recombinant receptors were purified utilizing nickel affinity chromatography. Soluble recombinant human CD16-Phe158 and CD16-Val158 were expressed without their C-terminal transmembrane domains and used native signal sequences. The recombinant CD16 proteins were purified using anti-CD16 affinity chromatography.

Below are the sequences of the Fc gamma receptors that were used for recombinant expression of soluble receptors. The osteonectin signal peptide sequences are underlined.

Cynomolgus CD16

(SEQ ID NO: 1)

MRAWIFFLLCLAGRALAMRAEDLPKAVVFLEPQWYRVLEKDSVTLKCQGA
YSPEDNSTRWFHNESLISSQTSSYFIAAARVNNSGEYRCQTSLSTLSDPV
QLEVHIGWLLLQAPRWVFKEEESIHLRCHSWKNTLLHKVTYLQNGKGRKY
FHQNSDFYIPKATLKDSGSYFCRGLIGSKNVSSETVNITITQDLAVSSIS
SFFPPGYQTGTETSQVAPAASHHHHHH
human CD32A-Arg131

(SEQ ID NO: 2)

MRAWIFFLLCLAGRALAAPPKAVLKLEPPWINVLQEDSVTLTCQGARSPE
SDSIQWFHNGNLIPTHTQPSYRFKANNNDSGEYTCQTGQTSLSDPVHLTV
LSEWLVLQTPHLEFQEGETIMLRCHSWKDKPLVKVTFFQNGKSQKFSRLD
PTFSIPQANHSHSGDYHCTGNIGYTLFSSKPVTITVQVPSMGSSSPMTGT
ETSQVAPAASHHHHHH
human CD32A-His131

(SEQ ID NO: 3)

MRAWIFFLLCLAGRALAAPPKAVLKLEPPWINVLQEDSVTLTCQGARSPE
SDSIQWFHNGNLIPTHTQPSYRFKANNNDSGEYTCQTGQTSLSDPVHLTV
LSEWLVLQTPHLEFQEGETIMLRCHSWKDKPLVKVTFFQNGKSQKFSHLD
PTFSIPQANHSHSGDYHCTGNIGYTLFSSKPVTITVQVPSMGSSSPMTGT
ETSQVAPAASHHHHHH
human CD32B

(SEQ ID NO: 4)

MRAWIFFLLCLAGRALAAPPKAVLKLEPQWINVLQEDSVTLTCRGTHSPE
SDSIQWFHNGNLIPTHTQPSYRFKANNNDSGEYTCQTGQTSLSDPVHLTV
LSEWLVLQTPHLEFQEGETIVLRCHSWKDKPLVKVTFFQNGKSKKFSRSD
PNFSIPQANHSHSGDYHCTGNIGYTLYSSKPVTITVQAPSSSPMTGTETS
QVAPAASHHHHHH
human CD64

(SEQ ID NO: 5)

MRAWIFFLLCLAGRALAQVDTTKAVITLQPPWVSVFQEETVTLHCEVLHL
PGSSSTQWFLNGTATQTSTPSYRITSASVNDSGEYRCQRGLSGRSDPIQL
EIHRGWLLLQVS RVFTEGEPLALRCHAWKDKLVYNVLYYRNGKAFKFFH
WNSNLTILKTNISHNGTYHCSGMGKHRYTSAGISVTVKELFPAPVLNASV
TSPLLEGNLVTLSCETKLLLQRPGLQLYFSFYMGSKTLRGRNTSSEYQIL
TARREDSGLYWCEAATEDGNVLKRSPELELQVLGLQLPTPVWFHTGTETS
QVAPAASHHHHHH
murine CD16-1

(SEQ ID NO: 6)

MRAWIFFLLCLAGRALAALPKAVVKLDPPWIQVLKEDMVTLMCEGTHNPG
NSSTQWFHNGRSIRSQVQASYTFKATVNDSGEYRCQMEQTRLSDPVDLIG
VISDWLLLQTPQRVFLEGETITLRCHSWRNKLLNRISFFHNEKSVRYHHY
KSNFSIPKANHSHSGDYYCKGSLGSTQHQSKPVTITVQDPATTSSTGTET
SQVAPAASHHHHHH
murine FcRIV

(SEQ ID NO: 7)

MRAWIFFLLCLAGRALAQAGLQKAVVNLDPKWVRVLEEDSVTLRCQGTFS
PEDNSIKWFHNESLIPHQDANYVIQSARVKDSGMYRCQTALSTISDPVQL
EVHMGWLLLQTTKWLFQEGDPIHLRCHSWQNRPVRKVTYLQNGKGKKYFH
ENSELLIPKATHNDSGSYFCRGLIGHNNKSSASFRISLGDPGSPSMFPPW
HQTGTETSQVAPAASHHHHHH
human FcRn

(SEQ ID NO: 8)

MRAWIFFLLCLAGRALAAESHLSLLYHLTAVSSPAPGTPAFWVSGWLGPQ
QYLSYNSLRGEAEPCGAWVWENQVSWYWEKETTDLRIKEKLFLEAFKALG
GKGPYTLQGLLGCELGPDNTSVPTAKFALNGEEFMNFDLKQGTWGGDWPE
ALAISQRWQQQDKAANKELTFLLFSCPHRLREHLERGRGNLEWKEPPSMR
LKARPSSPGFSVLTCSAFSFYPPELQLRFLRNGLAAGTGQGDFGPNSDGS
FHASSSLTVKSGDEHHYCCIVQHAGLAQPLRVELESPAKSGSGTGTETSQ
VAPAASHHHHHH
human CD16-Phe158

(SEQ ID NO: 9)

MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLFGSKNVSSETVNITITQGLAVSTIS
SFFPPGYQ
human CD16-Val158

(SEQ ID NO: 9)

MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLVGSKNVSSETVNITITQGLAVSTIS
SFFPPGYQ

### Cells

The Hodgkin's lymphoma cell line L540 (ACC-72) was grown in RPMI1640 (Mediatech, 10-040-CV) media containing 10% fetal bovine serum (Invitrogen, 02-4012DK).

### ADCC Assays

Europium ADCC Assay

L540 cells were employed in a modified ADCC assay that used a time resolved fluorescence detection method. Human peripheral blood mononuclear cells were purified from heparinized whole blood by standard Ficoll-paque separation. The cells were resuspended in RPMI1640 media containing 10% FBS and 50-200 U/mL of human IL-2 and incubated overnight at 37° C. The following day, the cells were collected and washed once in culture media and resuspended at 1×10⁷ cells/mL. Two million target L540 cells were incubated with 20 μM TDA reagent (Perkin Elmer) and 2.5 mM probenecid (Sigma) in 2 mL total volume for 20 minutes at 37° C. The target cells were washed three times in PBS with 2.5 mM probenecid and 20 mM HEPES. The cells were then resuspended to a final volume of 1×10⁵ cells/mL in probenecid containing culture media. For the final ADCC assay, 100 μL of labeled L540 cells were incubated with 50 μL of effector cells and 50 μL of antibody. The final target to effector ratio of 1:50 was selected. In all studies, human IgG1 isotype control was run and compared to anti-CD30 antibody. Other controls were: a) target and effector cells but no antibody, b) target cells with no effector cells, and c) wells containing target and effector cells in the presence of 3% Triton X-100 or Lysol® as 100% lysis. Following a 1 hour incubation at 37° C., 20 μL of the supernatants were collected into a flat bottom plate and mixed with 180 μL of europium substrate solution. The reaction was read with a Perkin Elmer Fusion Alpha TRF reader using a 400 μsec delay and 330/80 620/10 excitation and emission filters, respectively. The counts per second were plotted as a function of antibody concentration and the data was analyzed by non-linear regression, sigmoidal dose response (variable slope) using Prism software (San Diego, Calif.). The percent lysis was determined by the following equation:

% Lysis=(Sample CPS-no antibody CPS)/100% lysis CPS-No antibody CPS)×100.

⁵¹Cr Assay

Human peripheral blood mononuclear cells were purified from heparinized whole blood by standard Ficoll-paque separation. The cells were resuspended (at 1×10⁶ cells/mL) in RPMI1640 media containing 10% FBS and 50-200 U/mL of human IL-2 and incubated overnight at 37° C. The following day, the cells were collected and washed once in culture media and resuspended at 2×10⁷ cells/mL. Two million target L540 cells were incubated with 200 μCi ⁵¹Cr in 1 mL total volume for 1 hour at 37° C. The target cells were washed once, resuspended in 1 mL of media, and incubated at 37° C. for an additional 30 minutes. After the final incubation, the target cells were washed once and brought to a final volume of 1×10⁵ cells/mL. For the final ADCC assay, 100 μL of labeled L540 cells were incubated with 50 μL, of effector cells and 50 μL of antibody. The final target to effector ratio of 1:100 was selected. In all studies, human IgG1 isotype control was run and compared to wild type or variant antibodies. Other controls included: a) target and effector cells but no antibody, b) target cells with no effector cells, and c) wells containing target and effector cells in the presence of 3% Triton X-100 or Lysol® as 100% lysis. Following a 4 hour incubation at 37° C., the supernatants were collected and counted on a gamma Counter (Cobra II auto-gamma from Packard Instruments) with a reading window of 240-400 keV. The counts per minute were plotted as a function of antibody concentration and the data was analyzed by non-linear regression, sigmoidal dose response (variable slope) using Prism software (San Diego, Calif.). The percent lysis was determined by the following equation:

% Lysis=(Sample CPM-no antibody CPM)/100% lysis CPM-No antibody CPM)×100.

### Results

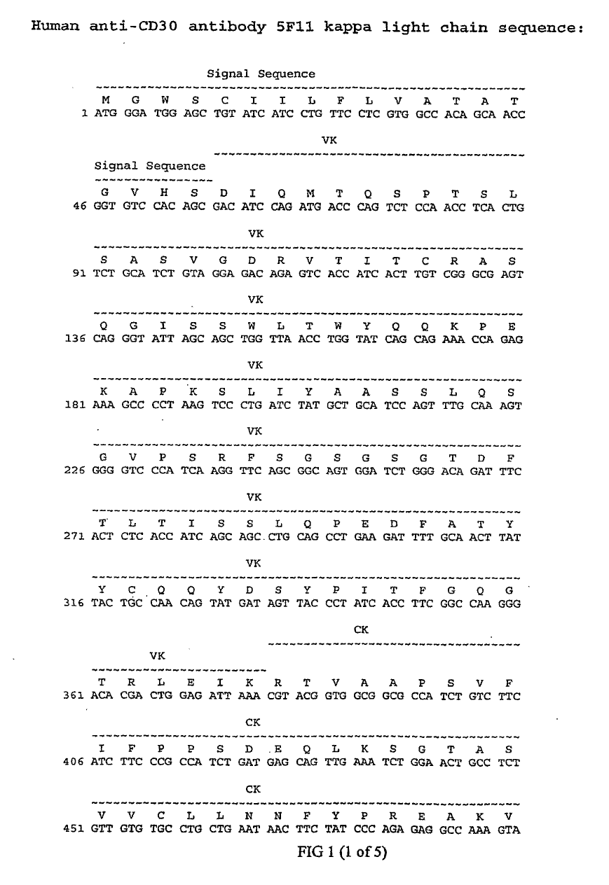

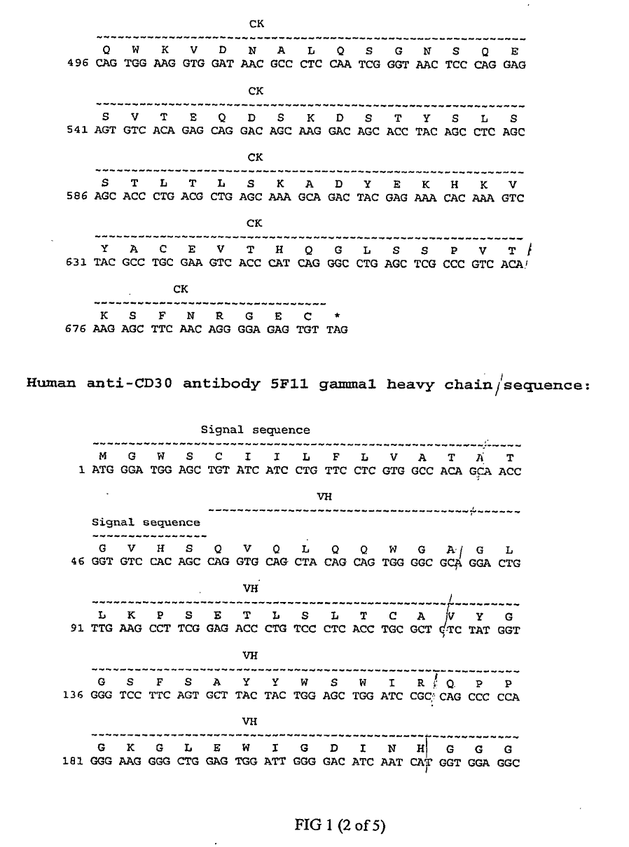

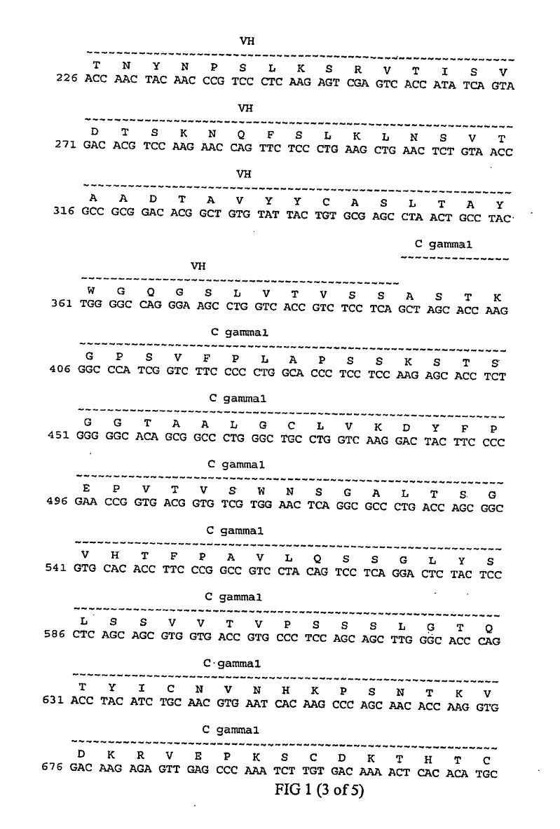

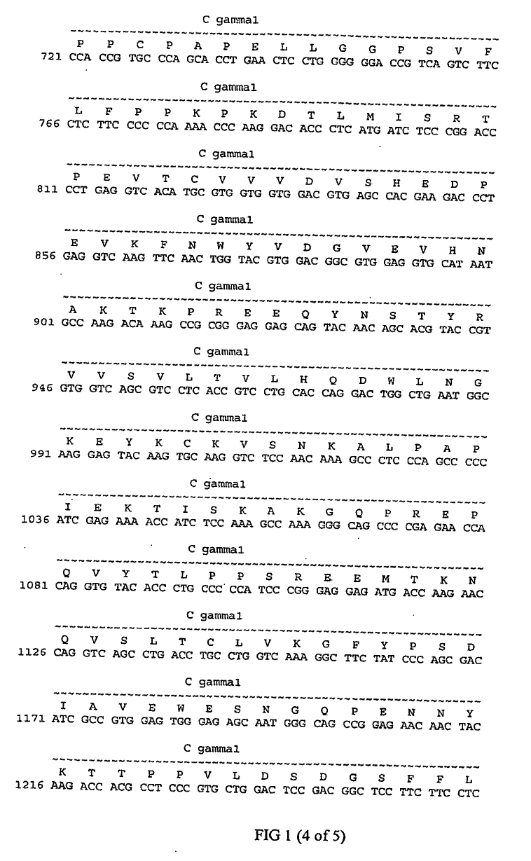

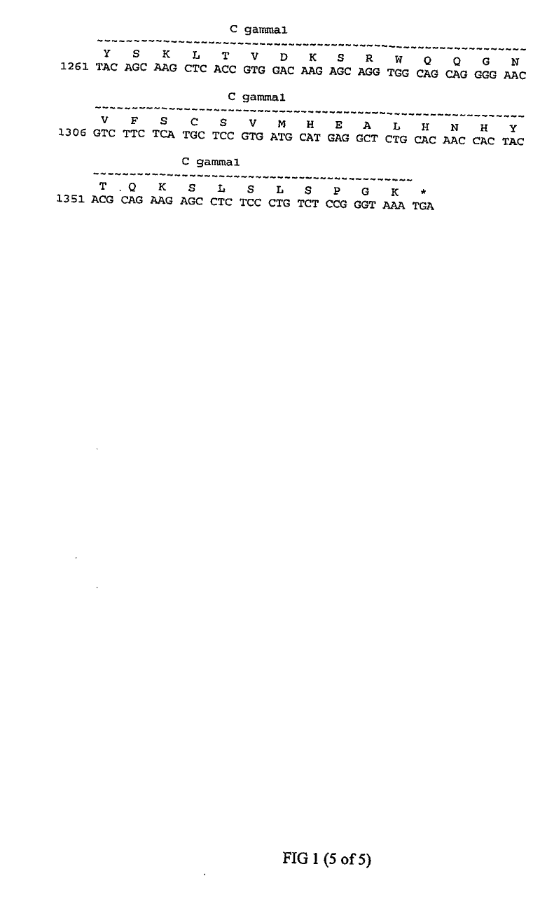

FIG. 1 presents the sequence of the anti-CD30 antibody (5F11) that was mutated to produce the variants of the invention. The heavy chain of this antibody is of the gammal f allotype. The light chain is a kappa light chain. The variable region sequences of the antibody are published in WO 03/059282. The mutagenesis of the antibody involved the constant region rather than the antigen-binding region.

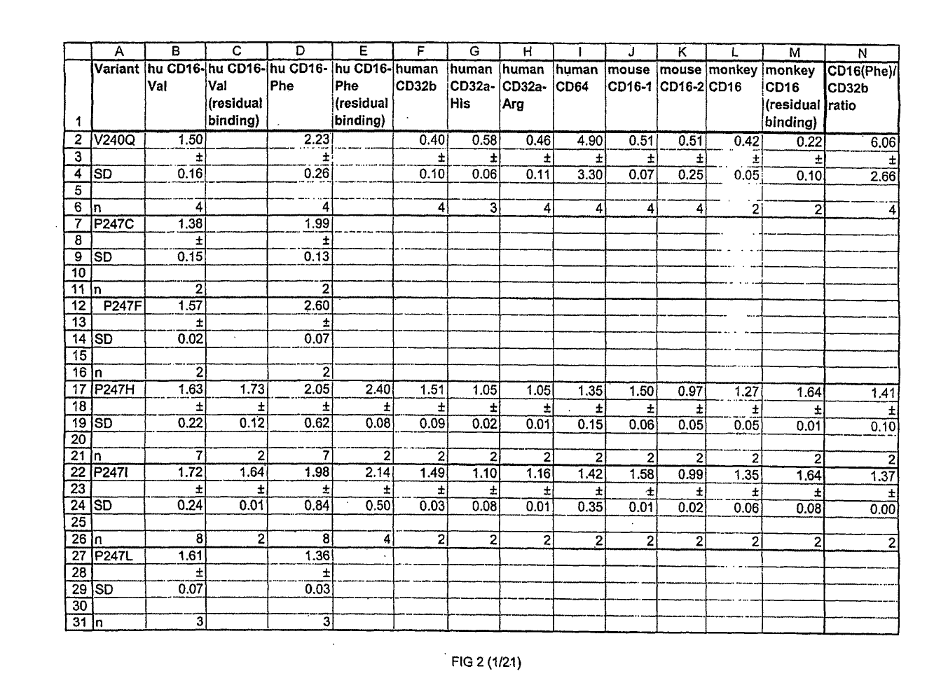

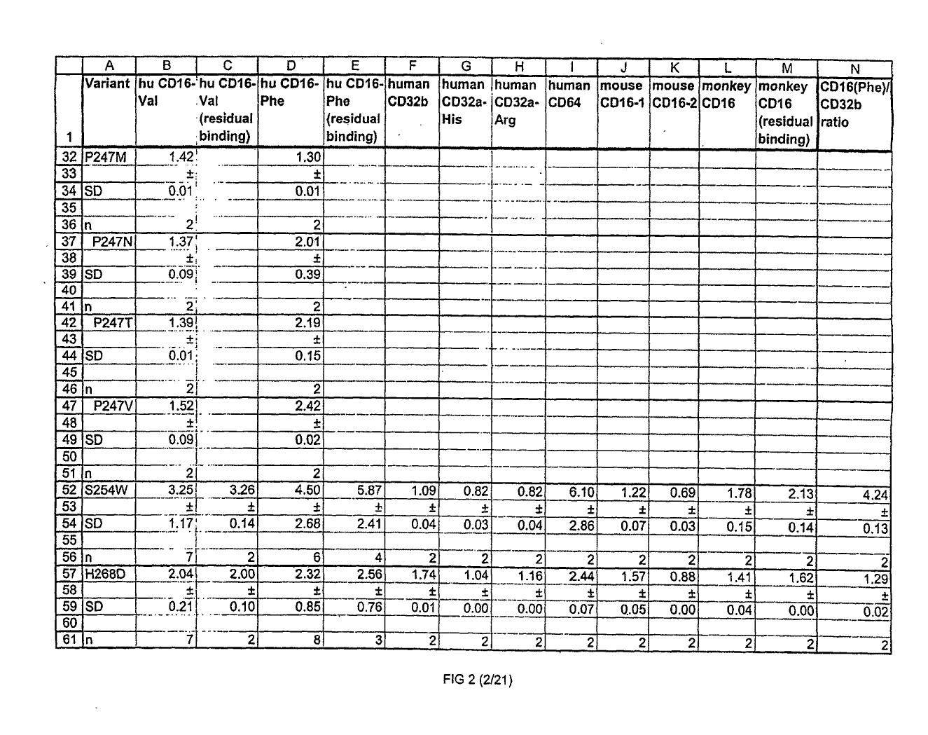

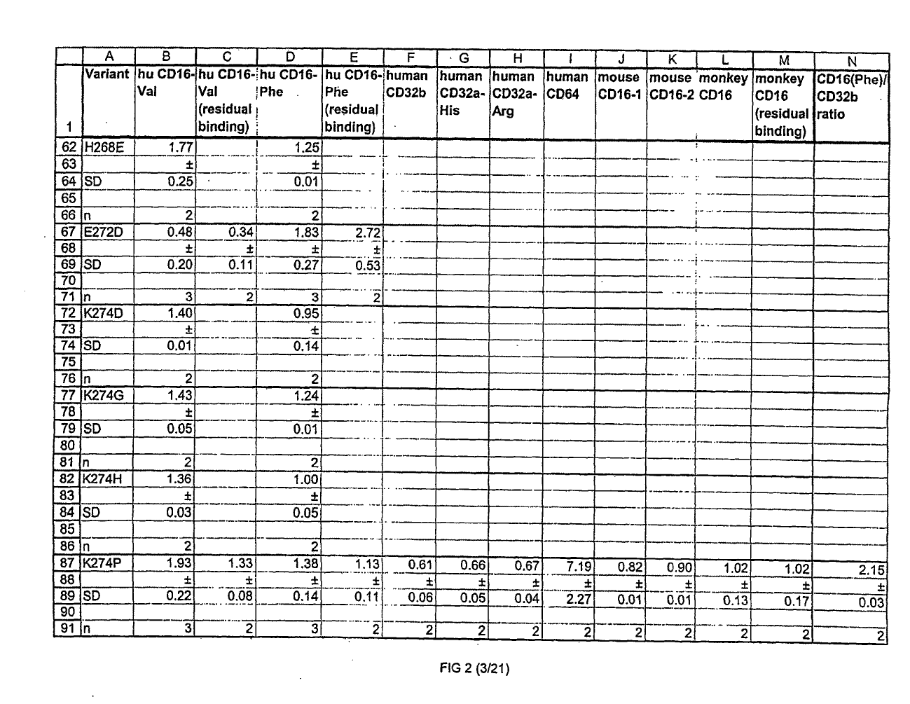

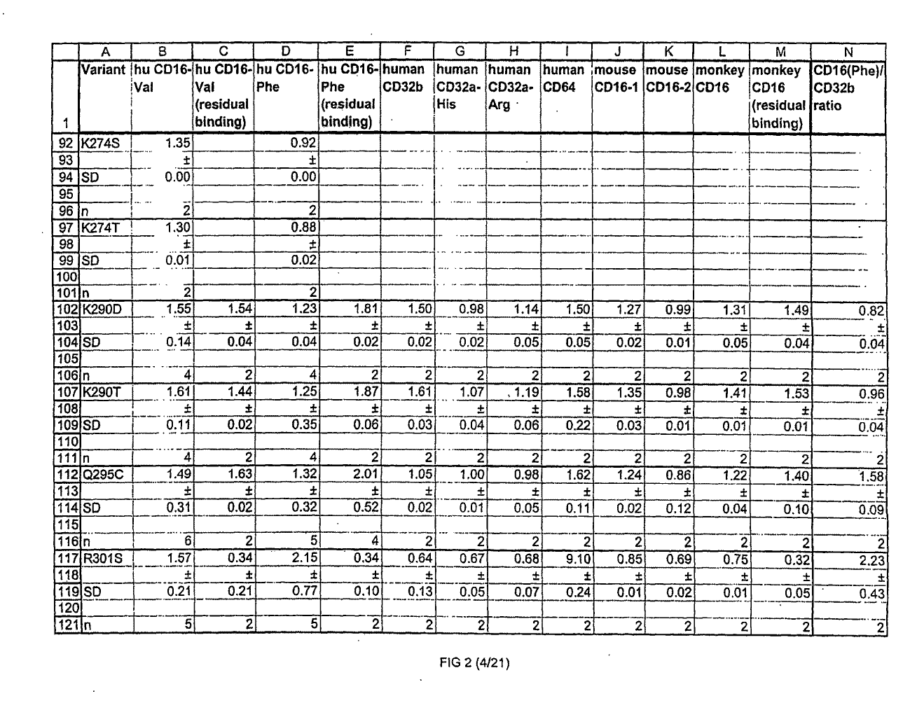

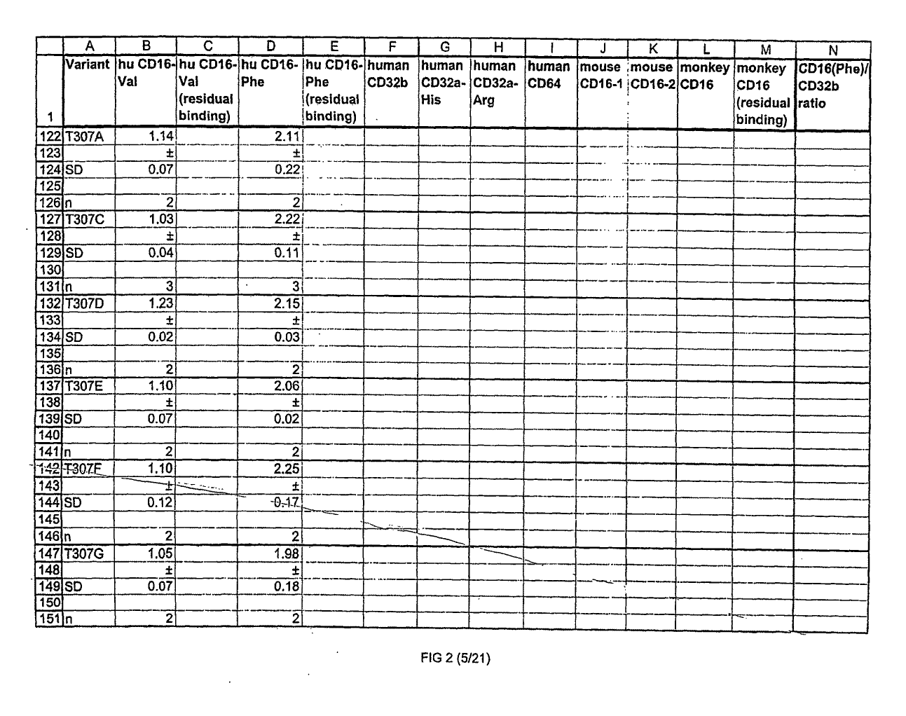

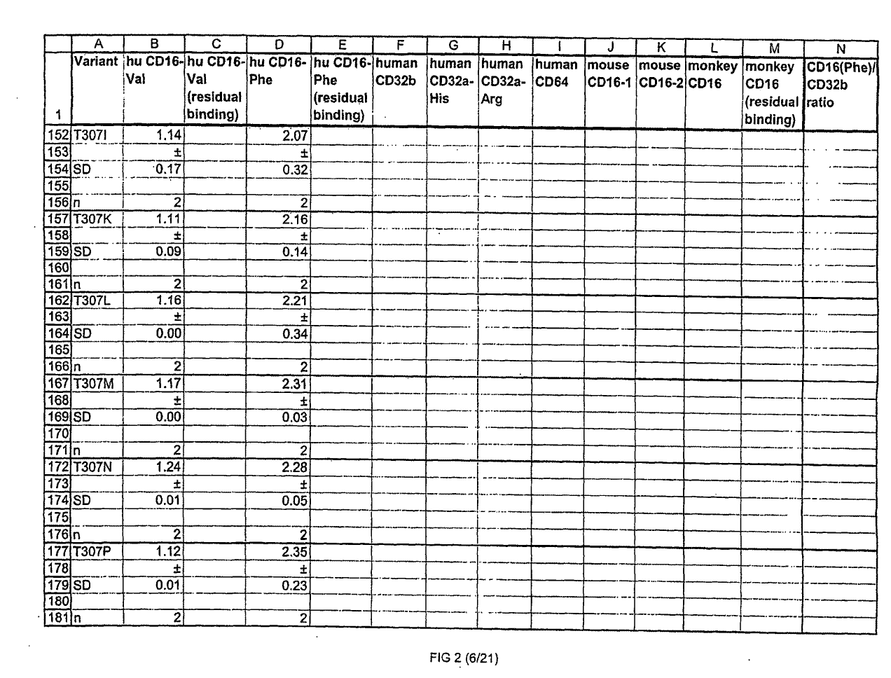

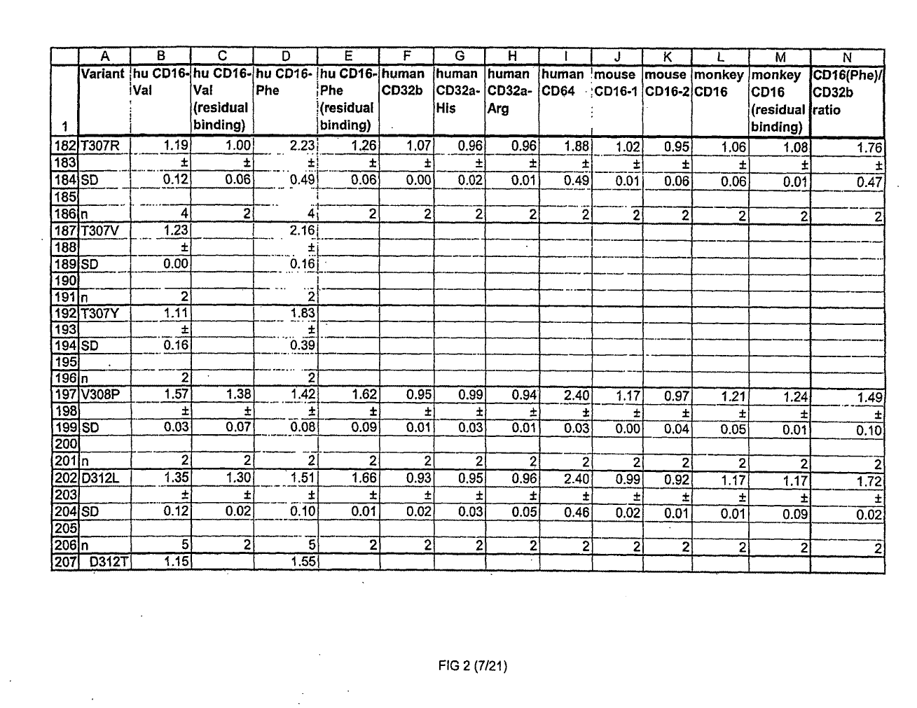

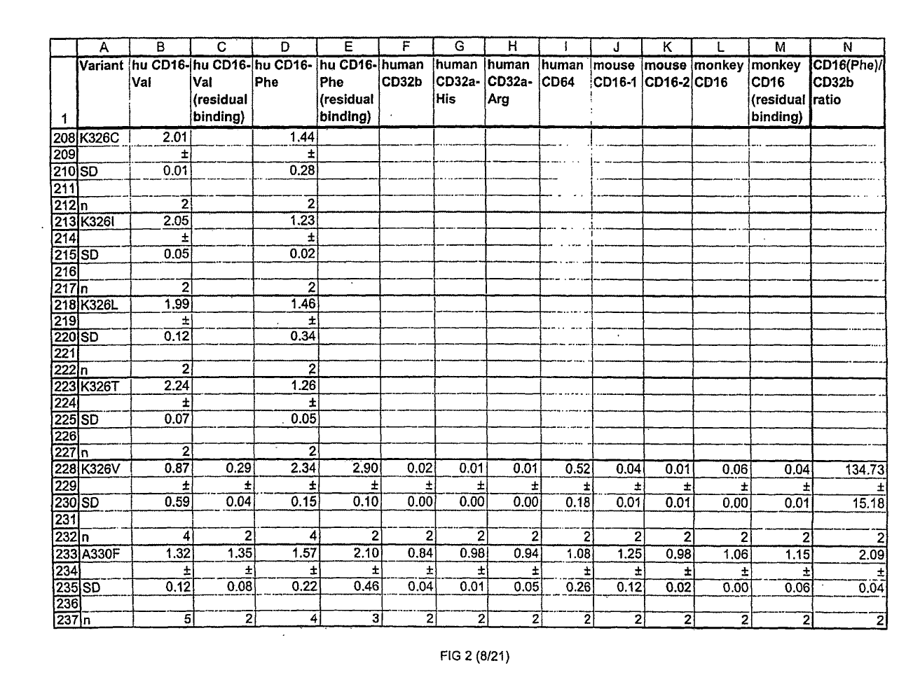

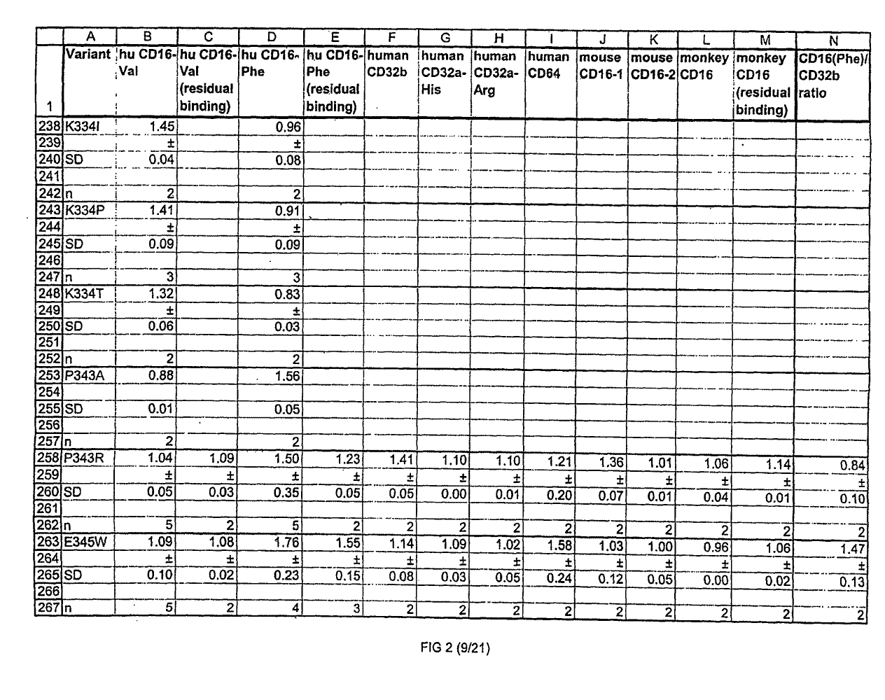

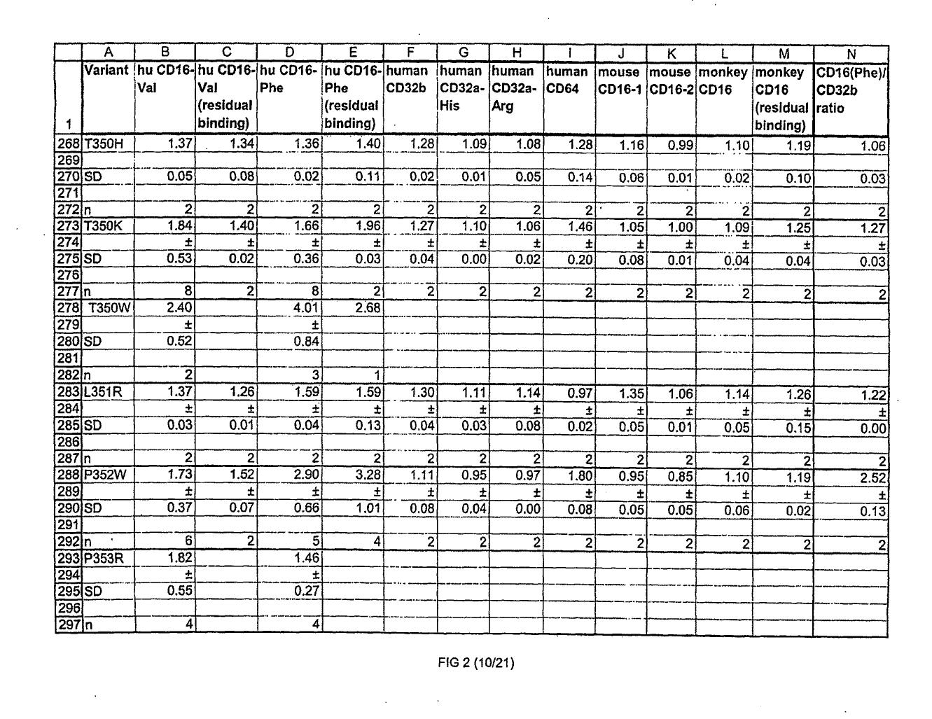

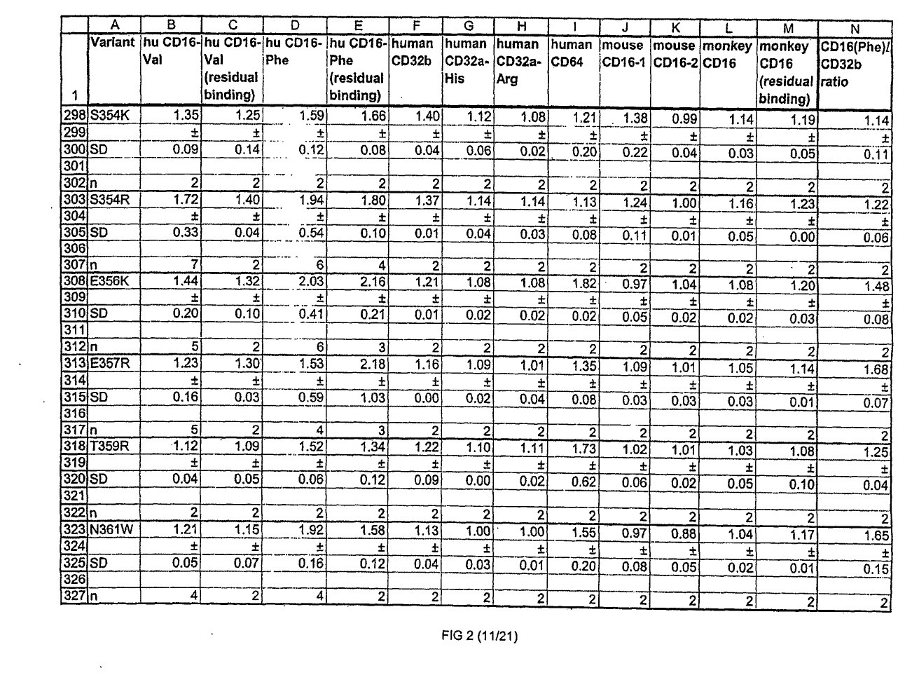

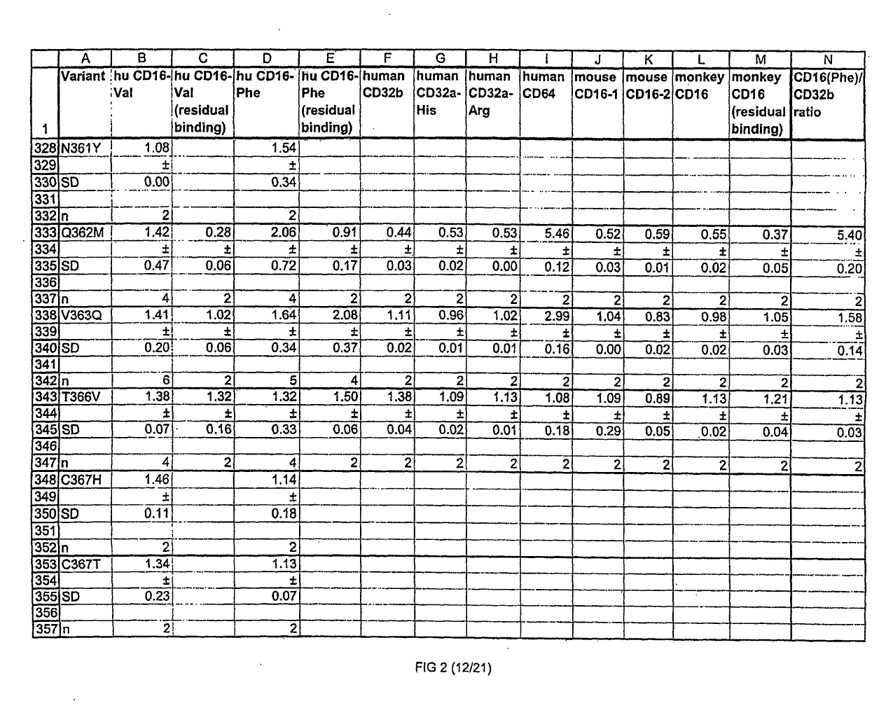

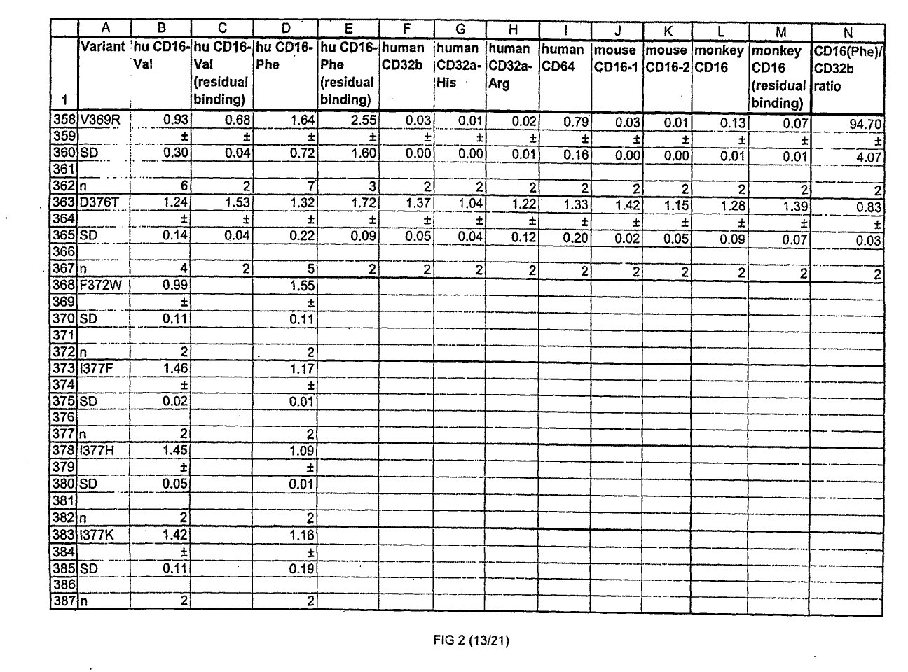

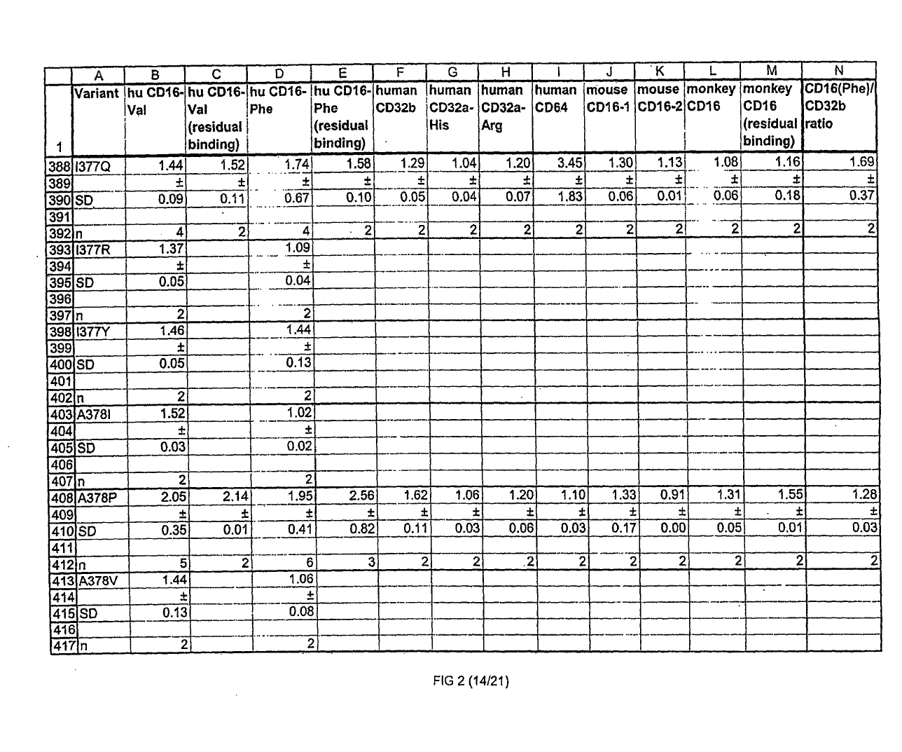

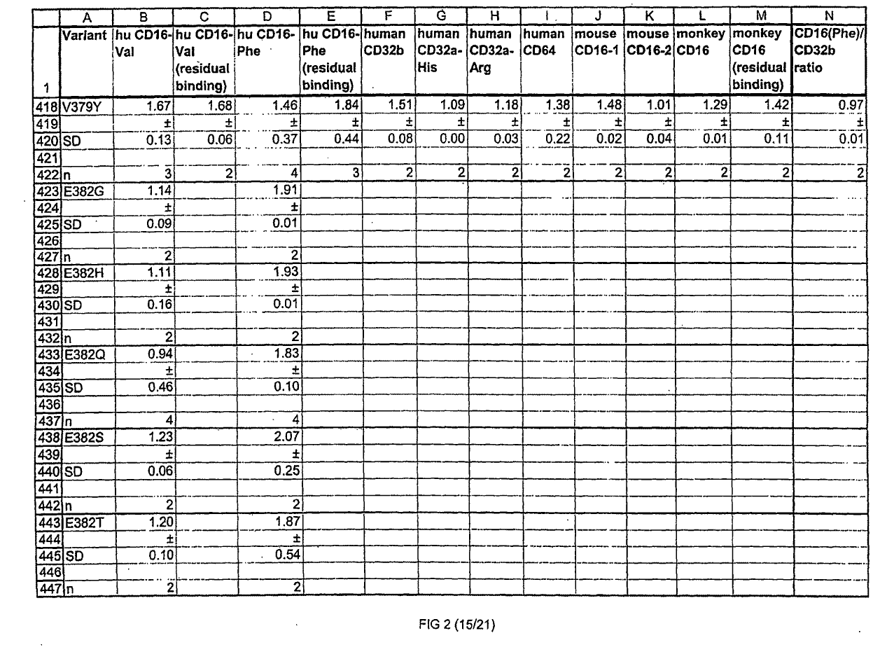

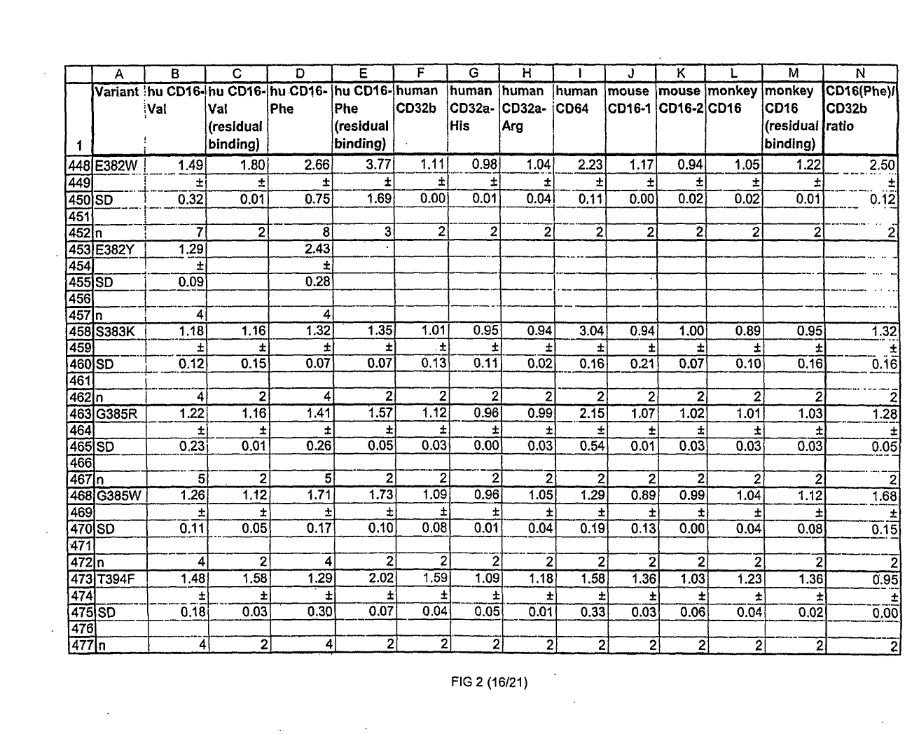

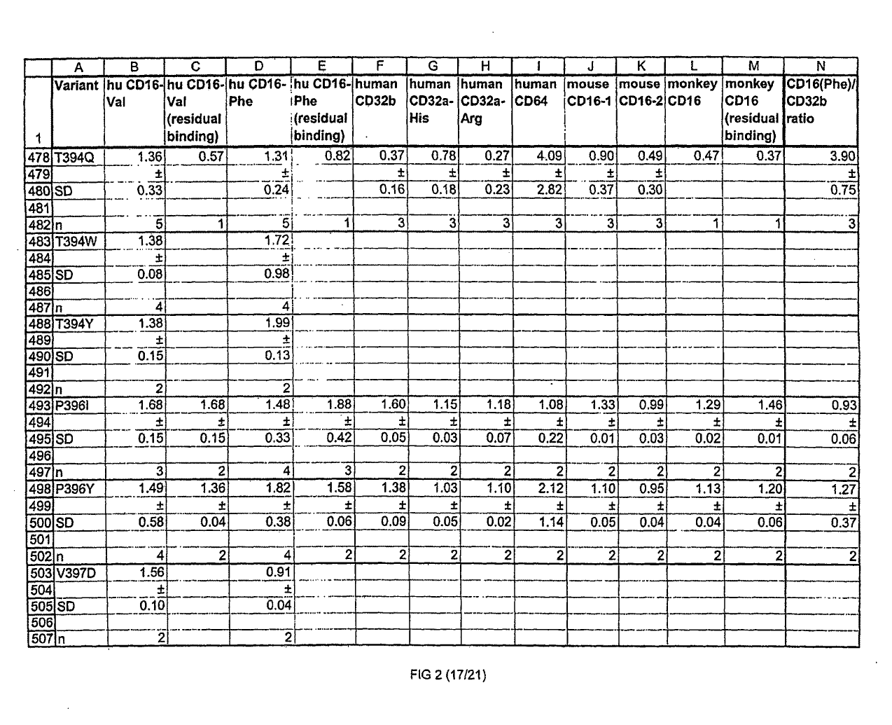

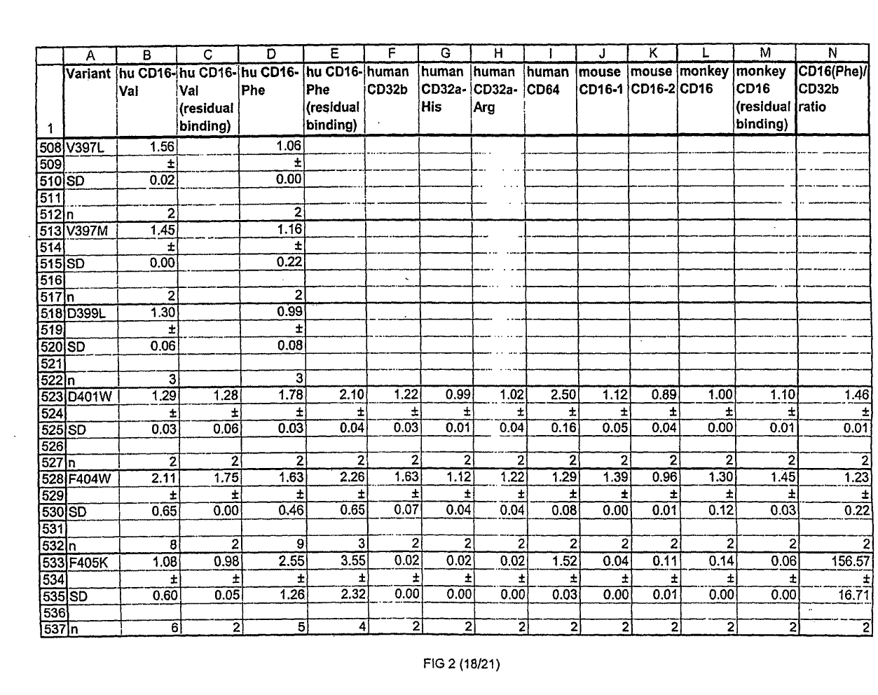

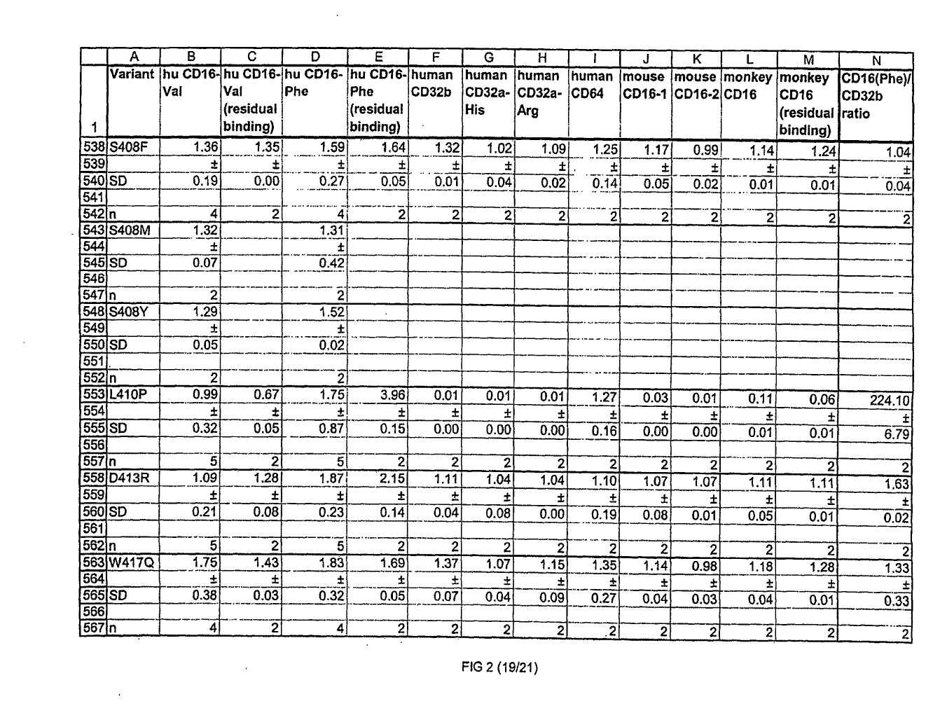

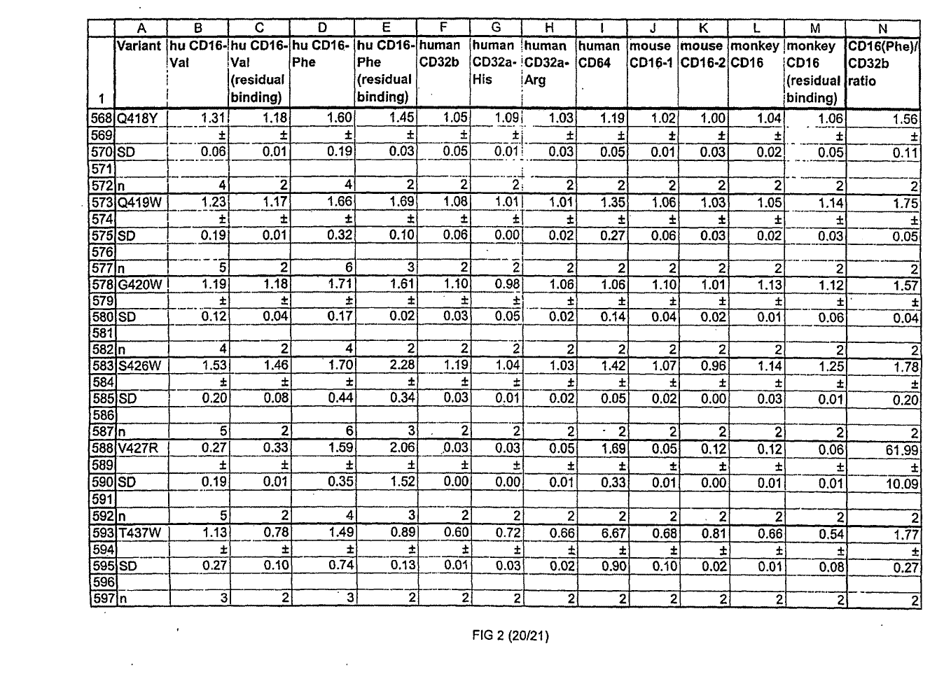

The following relates to the results presented in FIG. 2. In column A, antibody (Ab) variants of the invention are designated by their amino acid residue number (EU Rabat). The first letter refers to the wild type amino acid and the last letter refers to the variant amino acid. For example, V240Q indicates that the variant contains a glutamine (Q) at amino acid position 240 instead of a valine (V).

As depicted in columns B, D, F, G, H, I, J, K, and L, the Ab variants were assayed and compared to the wild type using a binding assay. This binding assay measures the binding of the Ab variant to each of the individual Fc receptors listed at the top of the respective column. The average values (±standard deviation, SD) listed in the table are derived from a collection of binding results (n replicates) expressed as the ratio of the signal produced by the Ab variant divided by the wild-type anti-CD30 Ab signal. For example, a ratio of 1 indicates that the Ab variant bound to a particular Fc receptor (listed at the top of the column) and gave a signal equal to the wild type Ab. A ratio of 2 indicates that the Ab variants bound to a particular Fc receptor (listed at the top of the column) and gave a signal 2-fold greater than the wild type Ab.

Columns C, E, and M provide relative residual binding measurements as measured using a binding assay. The relative residual binding assay measures the variant still bound to each of the individual Fc receptors (listed at the top of the column) 1 hour after all the assay reagents are diluted 10-fold. The average values (±SD) listed in the table are derived from a collection of binding results (n replicates) expressed as the ratio of the signal produced by the Ab variant divided by the wild-type anti-CD30 Ab signal. For example, a ratio of 1 indicates that the Ab variant bound to a particular Fc receptor (listed at the top of the column) and gave a signal equal to the wild type Ab. A ratio of 2 indicates that the Ab variants bound to a particular Fc receptor (listed at the top of the column) and gave a signal 2-fold higher than the wild type Ab.

Column N provides a mathematical ratio generated by dividing the huCD16-Phe ratio value in Column D by the human CD32b ratio value in Column F. A large ratio indicates higher antibody binding to huCD16-Phe relative to huCD32b, a presumed binding characteristic of antibodies having enhanced ADCC function.

Each Ab variant contains a set of 3 rows indicating the following values: The first row represents the average binding ratio values corresponding to each Fc receptor. Ab variants listed in this table generally have an average huCD16-Val binding ratio ≧1.3 (Col B) or a huCD16-Phe binding ratio ≧1.5 (Col D). The second row represents the standard deviations (SD) of the binding ratio values corresponding to each Fc receptor. The third row represents the total number of Ab variant samples (n replicates) that have been individually screened on the binding assay that corresponds to each Fc receptor.

It is of interest that variants A330F and P247V exhibited increased human CD16 binding since these variants have been reported by others (e.g., U.S. published application No. 2004/0123101) to have reduced binding to the same receptor.

Single substitution antibody variants were also tested in ADCC assays and the activities were compared to wild type antibody. The variants were tested at 2 concentrations, 0.5 μg/mL and 0.01 μg/mL, and percent lysis was calculated. The Delfia® ADCC assay was run initially followed by the ⁵¹Cr release assay. In general, the results from the two assays were similar. Antibodies with substitutions that induced % lysis greater than the wild type antibody are shown in FIG. 3. Eight to ten of the single substitutions were selected for incorporation into reassembly libraries.

### Example 2

### Analysis of Fc Region Variants with Multiple Substitutions

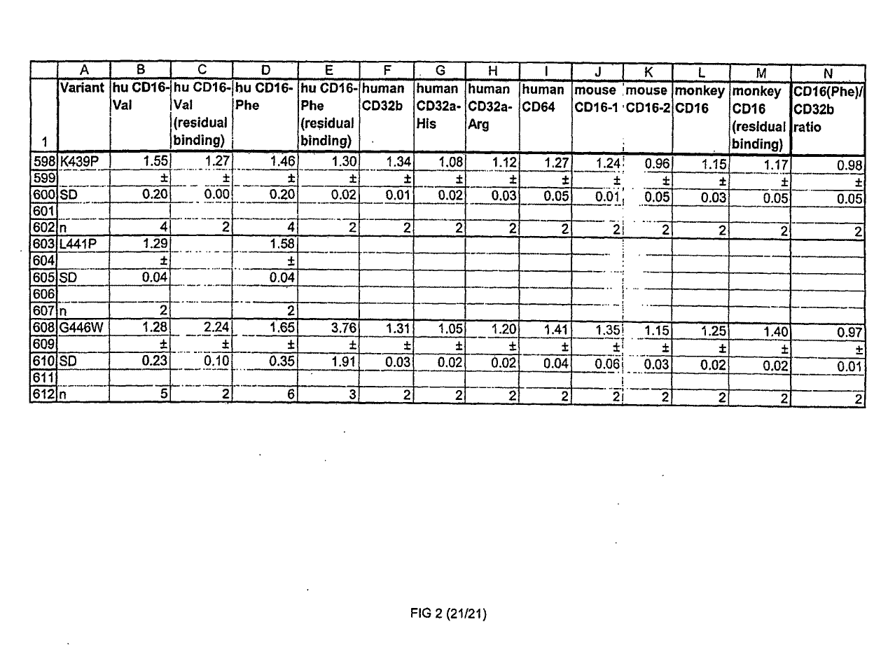

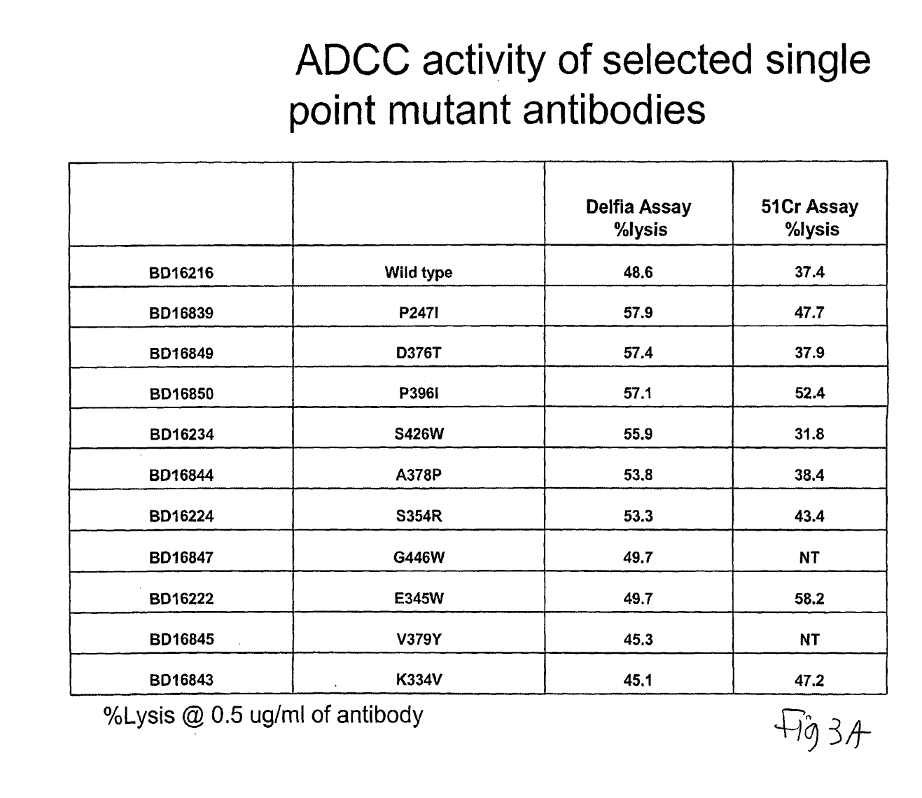

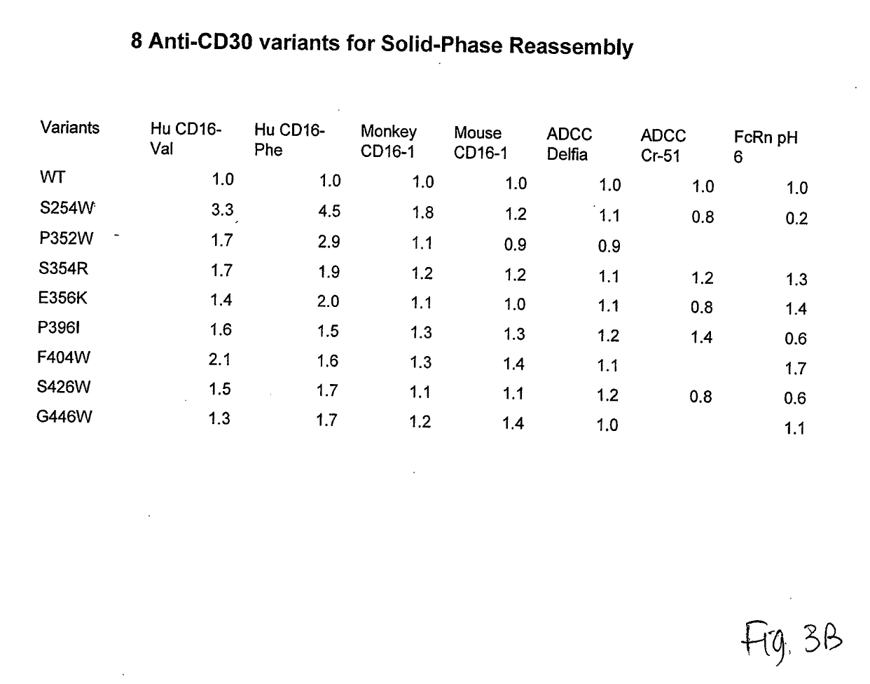

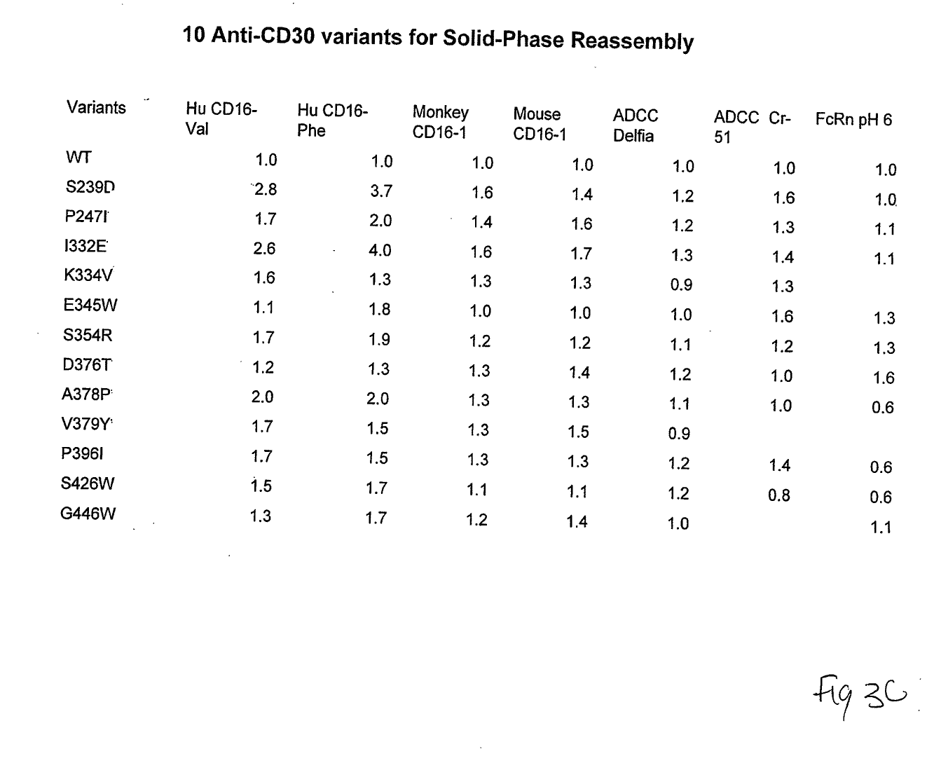

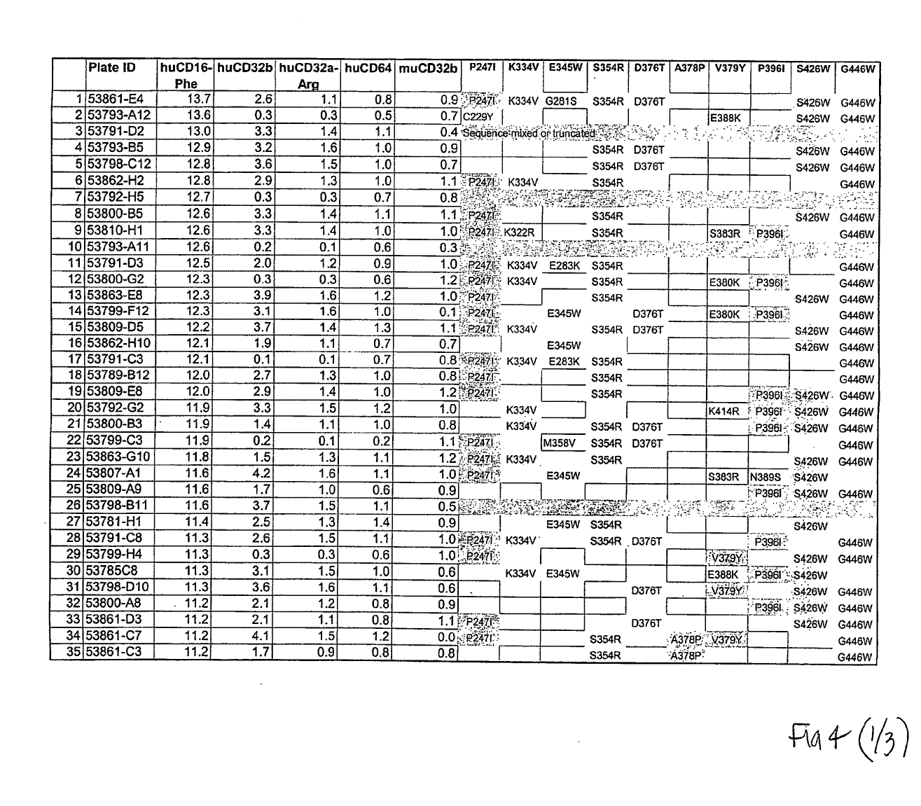

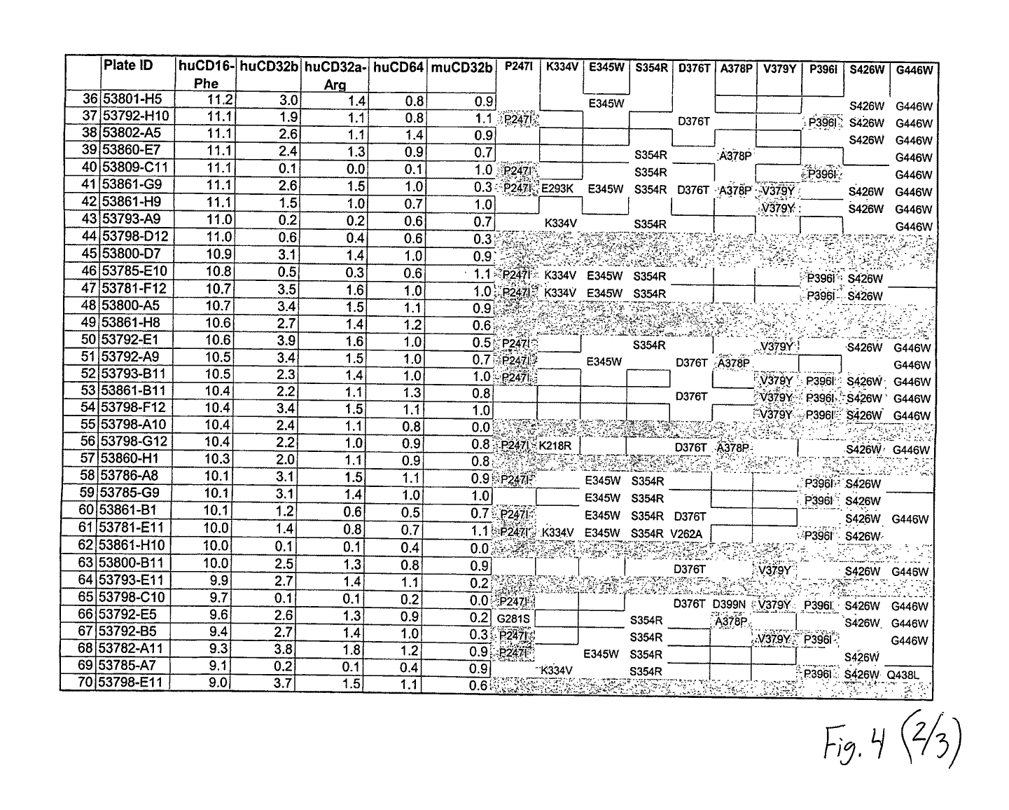

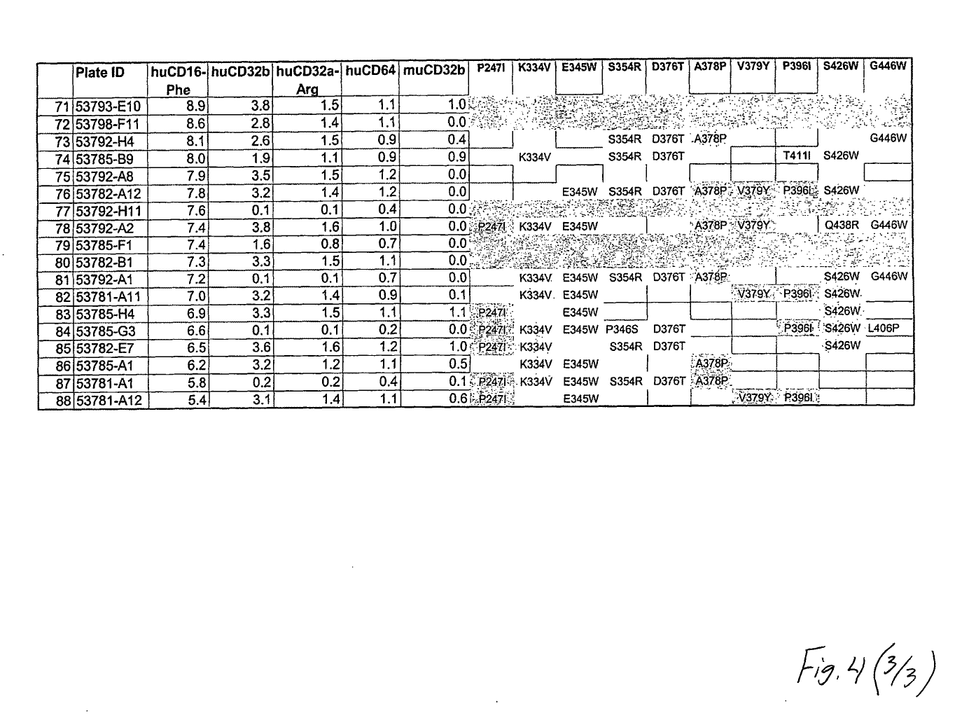

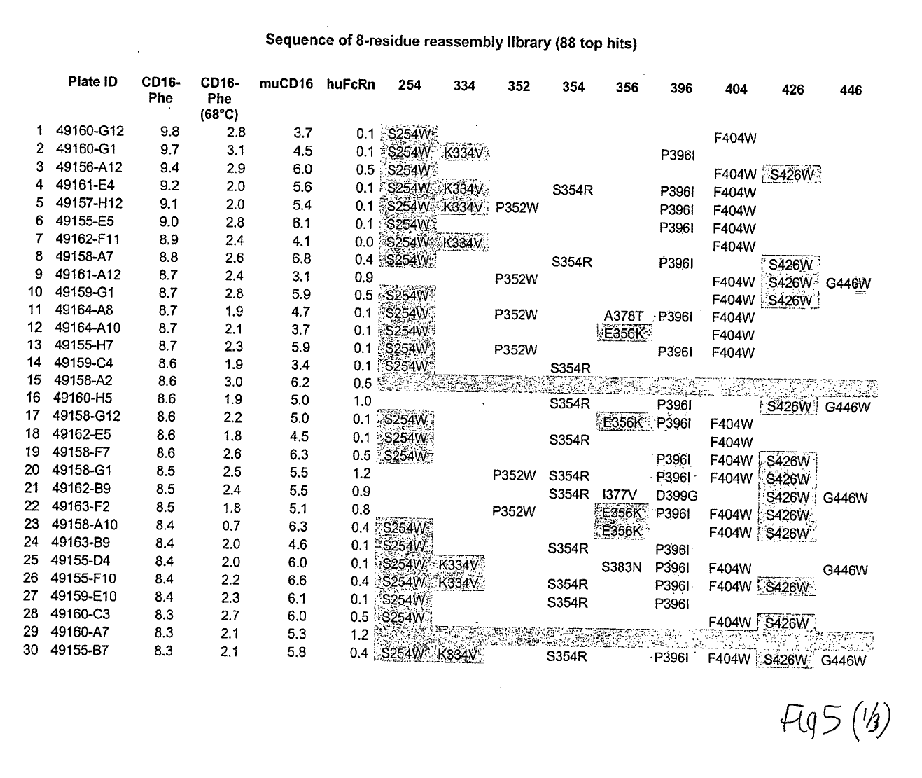

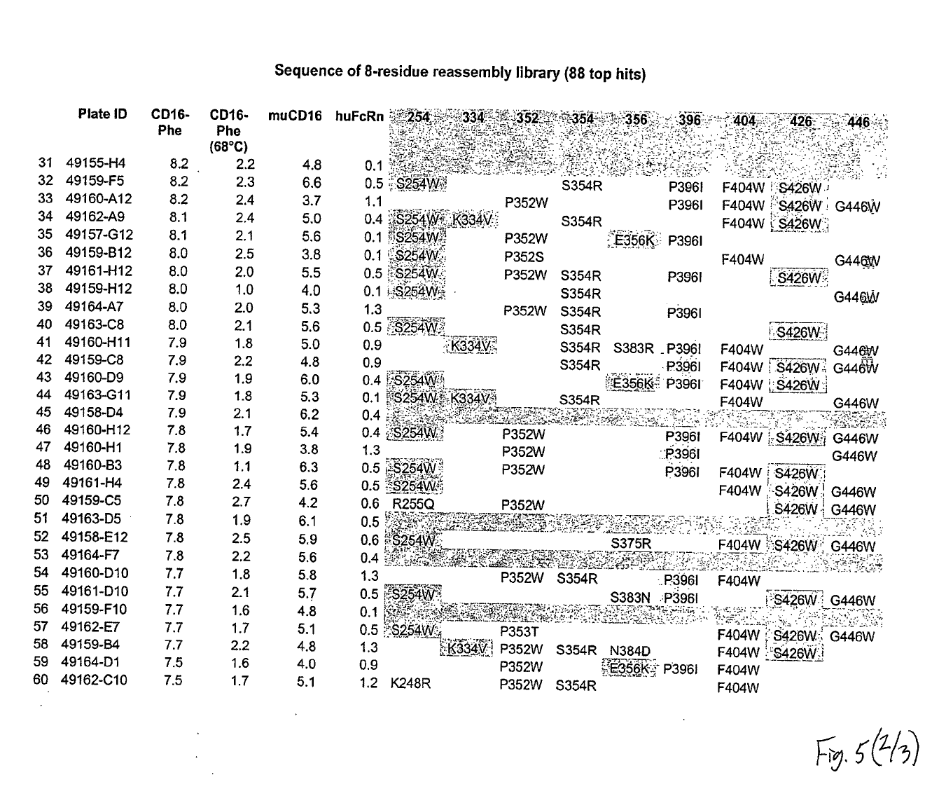

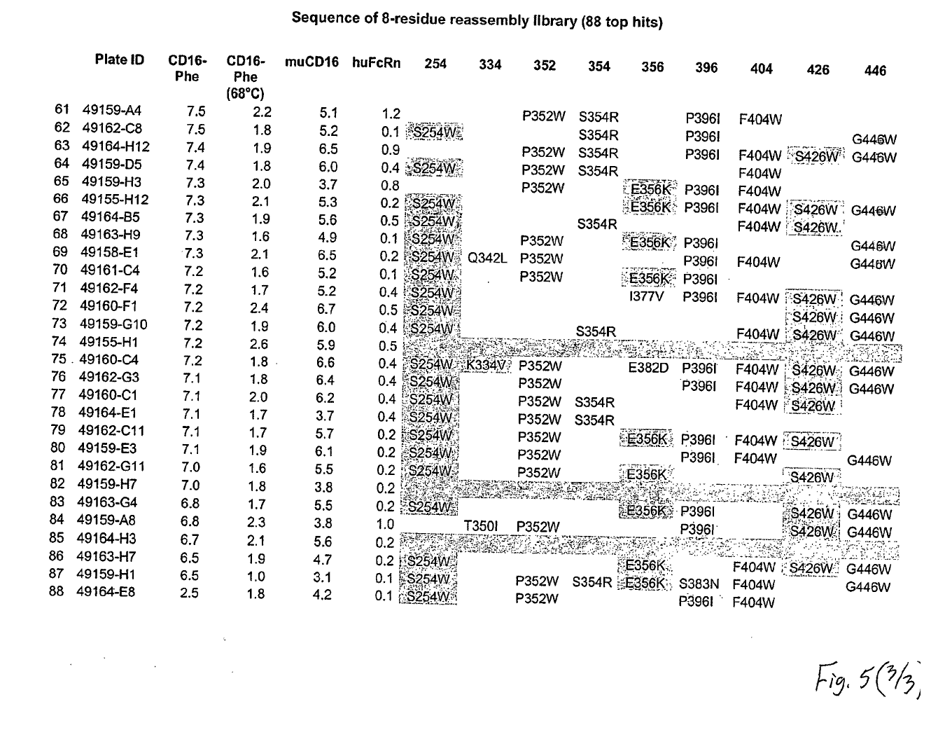

A subset of substitutions from antibodies with improved CD16 binding (Example 1) were used to prepare a library of antibodies with two or more substitutions. One library was prepared with substitutions at 8 different positions (the 8 residue library), and another library was prepared with substitutions at 10 different positions (the 10 residue library). The libraries were screened in in vitro binding assays and ADCC assays in a manner similar to that described in Example 1 (see FIGS. 4-7). For cell lysis, L540 cells and a Delfia ADCC assay were used. Generally, the assay (n=4) was run at 0.5 μg/mL and 0.01 μg/mL in triplicate. Controls generally included variants with corresponding single substitutions, a variant with S239D, S298A, and I332E (“293 Mut I”), and parental (“wild type”) CD30 monoclonal antibody (BD16216).

Based on the data in FIG. 6B, positions with substitutions resulting in the greatest enhancement of percent lysis were: 292, 297, 304, 310, 314, 315, 316, 320, 321, and 322, and those with highest mean percent lysis at 0.5 μg/mL:

were: 314, 315, 316, 320, 321, 322, 364, 366, 367, and 392. Six of those ten positions were identified in the 10 residue library and 4 were from the 8 residue library. Common positions for the greatest enhancement of lysis and mean percent lysis were: 292, 314, 315, 316, 320, 321, and 322. The top ten antibodies with altered Fc regions based on both criteria are present in antibodies BD20321, BD20292, BD20316, BD20320, BD20322, BD20315, BD20314, BD20304, BD20364, and BD20366.

To determine whether there was a correlation between ADCC results and huCD16-Val or -Phe binding results, certain antibodies with 1, 2 or 3 substitutions in the Fc region were selected for study (FIG. 7). Interestingly, antibodies with one or more substitutions in the Fc region with the highest CD16 binding were not as likely to be those with the most enhanced ADCC (FIG. 7A). In contrast, antibodies with one or more substitutions in the Fc region with poor CD16 binding did not have significantly enhanced ADCC. One explanation for these observations may be that even if the Fc binds the receptor, side chain interactions may be involved in enhancing ADCC. The results for huCD16-Phe binding showed a better correlation with ADCC than huCD16-Val binding (FIGS. 7B-C).

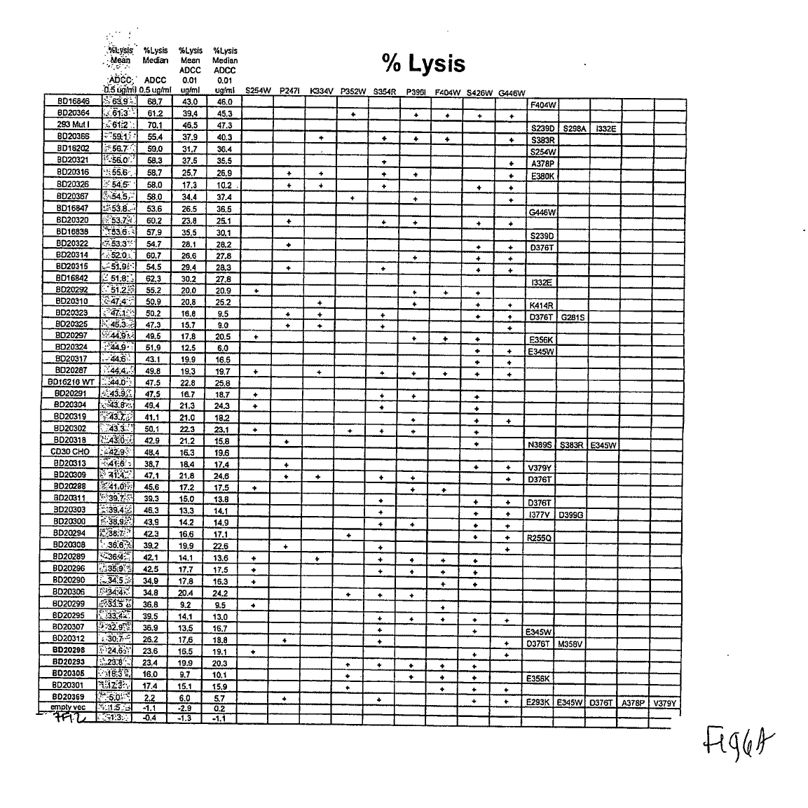

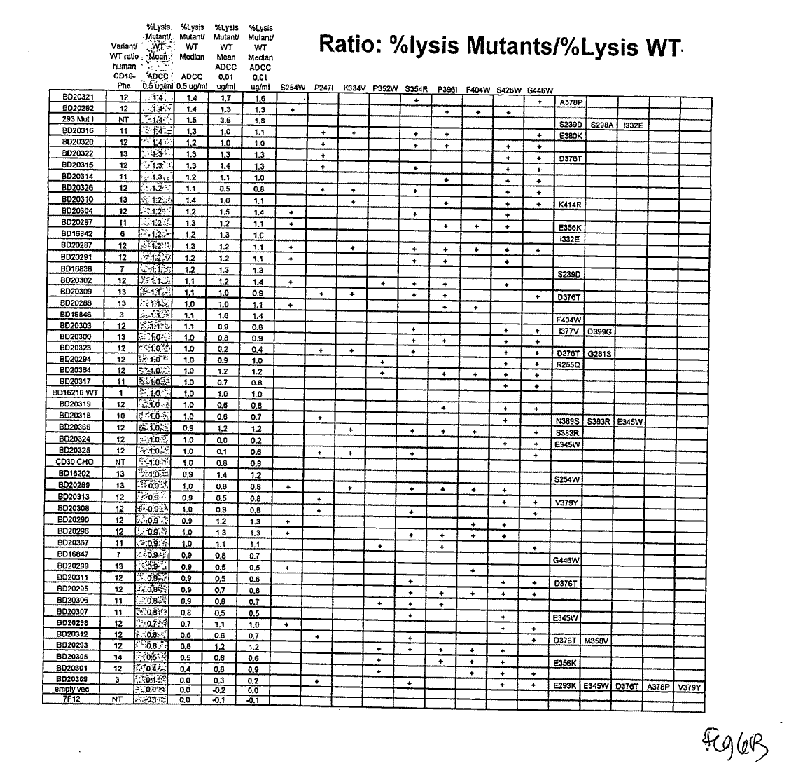

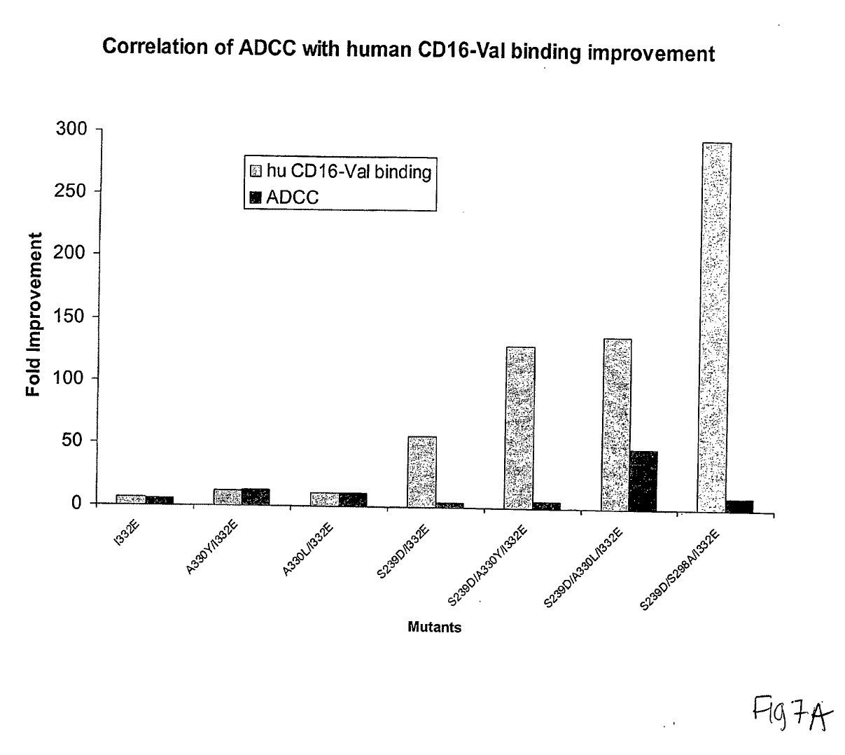

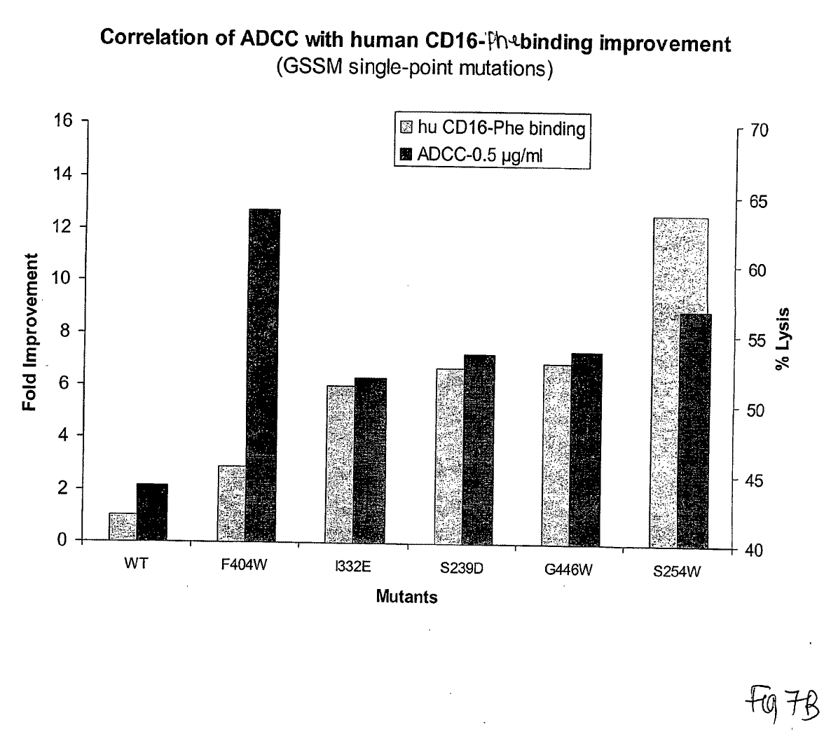

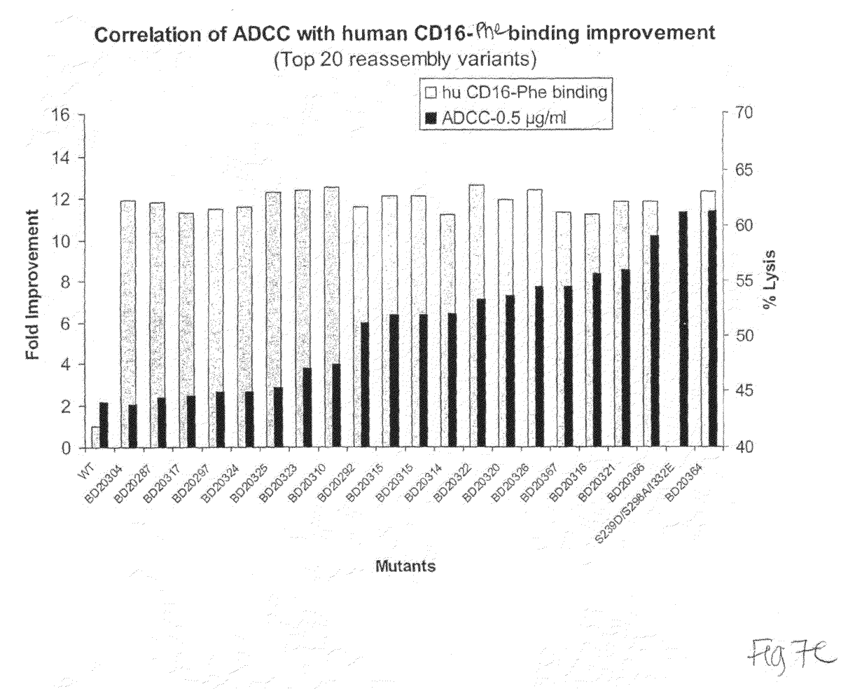

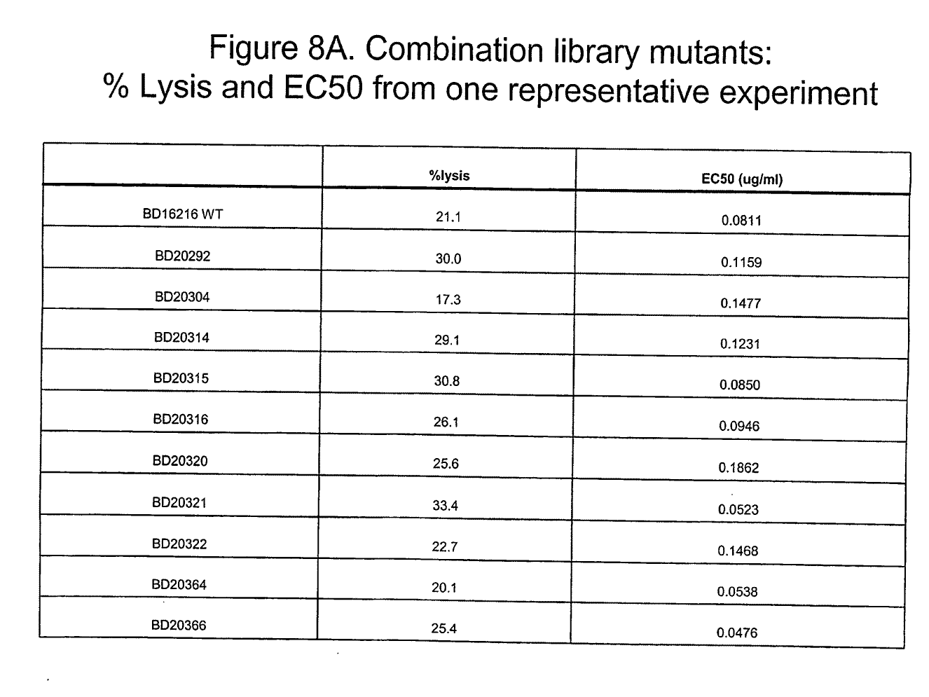

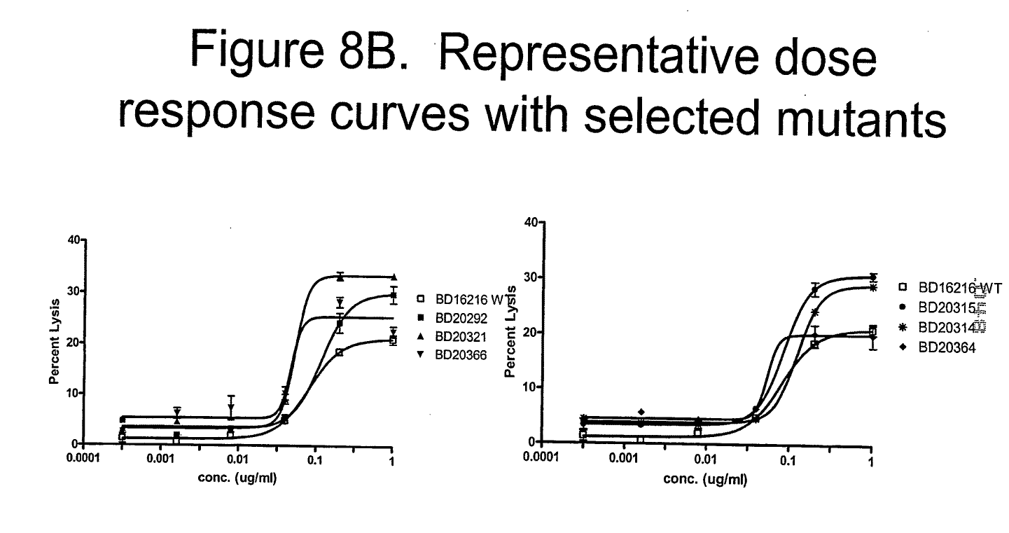

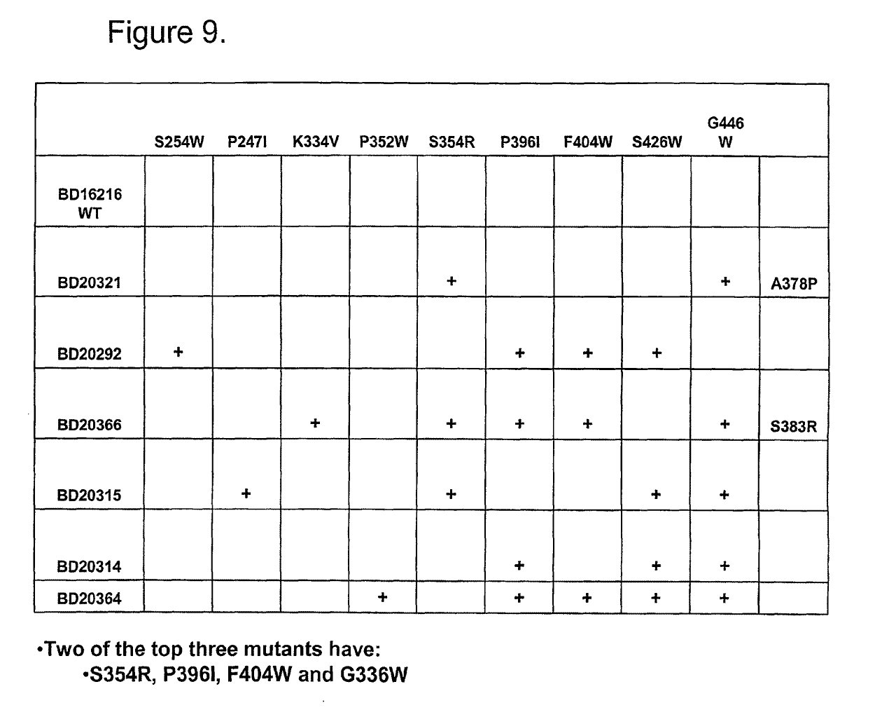

The percent lysis and EC50 data from one of four representative experiments are shown in FIG. 8A. Based on the results of the four experiments, six antibodies with substitutions in the Fc region were selected and the dose response curves for each of those antibodies compared to wild type antibody (FIG. 8B). The selection was based on a combination of improvement in lysis and EC50 data, as well as consistency from experiment to experiment. Based on the data, the following substitutions in combination with other substitutions have the most significant impact on ADCC enhancement: S354R, P396I, F404W and G336W. Two of the top three variants (FIG. 9) had the following substitutions: S354R, P396I, F404W, and G336W. Antibodies with this combination may result in improvement in both efficacy and potency.

### Example 3

### 3D Analysis of Fc-CD16 Interactions

Three dimensional models of Fc-CD16 interaction were analyzed for the location of amino acid residues that demonstrated different patterns of CD16 binding when amino acid substitutions were engineered at that position. CD16 shows an asymmetrical pattern of binding to the two arms of the Fc domain. Fc residues were identified as “hits” if one or more substitutions at that position resulted in increased CD16 binding, as “tolerant” if substitutions at that position had little effect on CD16 binding, as “intolerant” if almost all substitutions at that position resulted in decreased CD16 binding, and as “limited” if some substitutions at that position resulted in decreased CD16 binding. The intolerant residues primarily clustered in close proximity to the Fc-CD16 binding interface. The hits, residues that have identified mutations that increase CD16 binding, primarily clustered in three areas on the Fc. The first hit cluster is at residues that are located in area adjacent to the intolerant residues, and these may influence the interaction of the residues that are in contact with CD16. The second and third hit clusters are located at the CH2-CH3 elbow and the CH3-CH3 domain interface, respectively. The second and third hit clusters are distal from the CD16 binding site on the Fc domain and may increase the binding to CD16 by altering the angles or rotation of the Fc thereby influencing the interaction with CD 16.

### Example 4

### Analysis of Fc Region Substitutions Altering FcRn Binding

FcRn binding by single and multiple substitution variants was also assessed. The fold difference in binding as compared to wild type anti-CD30 (unaltered Fc) was measured in luminex assays. FcRn binding at 7.4 for all antibodies was 0 to 0.1. The results at pH 6 for single substrate are shown in Table 1.

TABLE 1

| | FcRn binding |
|---|---|
| Mutation | at pH 6 |
| None (wt) | 1 |
| E345W | 1.3 |
| P396I | 1.4 |
| P247I | 1.1 |
| S354R | 1.3 |
| A378P | 0.6 |
| D376T | 1.6 |
| S426W | 0.6 |
| E356K | 1.4 |
| S254W | 0.2 |
| F404W | 1.7 |
| G446W | 1.1 |

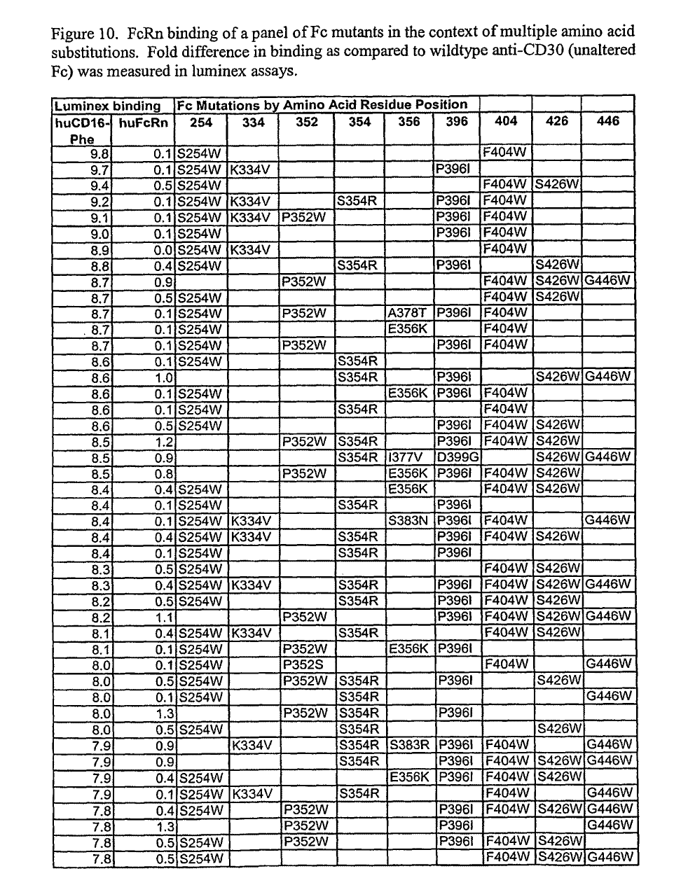

The S254W substitution resulted in a dramatic decrease in FcRn binding as a single substitution (Table 1) and in combination with other Fc region substitutions (FIG. 10). Altered Fc regions with changes in binding to human FcRn may result in changes in the half-lives of immunoglobulins containing the altered Fc regions. Engineering antibodies with altered half lives may have benefit for therapeutic applications, including antibodies with increased half lives that prolong activity and antibodies with decreased half lives that increase clearance of antibodies with undesirable prolonged exposure properties, such as radiolabeled antibodies.

### REFERENCES

Molecular Cloning: A Laboratory Manual (Sambrook et al., 3rd Ed., Cold Spring Harbor Laboratory Press, (2001).
Harlow and Lane, Antibodies: A Laboratory Manual, Cold Spring Harbor Laboratory Press, New York, 1988
Kabat et al., Sequences of Proteins of Immunological Interest, 5th Ed., United States Public Health Service, National Institutes of Health, Bethesda (1991)
Carter et al., Nucleic Acids Res., 13:4431 (1985)
Kunkel et al., Proc. Natl. Acad. Sci. USA, 82:488 (1987)
Higuchi, in PCR Protocols, pp. 177-183 (Academic Press, 1990)
Vallette et al., Nuc. Acids Res., 17:723 (1989)
Wells et al., Gene, 34:315 (1985)
Gazzano-Santoro et al., J. Immunol. Methods, 202:163 (1996)
Green et al., Nature Genet., 7:13 (1994)
Lonberg et al., Nature, 368:856 (1994)
Taylor et al., Int. Immun., 6:579 (1994)
McCafferty et al., Nature, 348:552 (1990)
Johnson and Chiswell, Current Opinion in Structural Biology, 3:5564 (1993)

While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

All documents, including but not limited to publications, patents and patent applications, cited herein are herein incorporated by reference.

1. An antibody comprising an altered Fc region, wherein the altered Fc region comprises at least one amino acid substitution relative to a parent IgG Fc region, wherein the at least one substitution is at a position corresponding to position 240, 247, 254, 268, 272, 274, 290, 295, 301, 307, 308, 312, 326, 330, 334, 343, 345, 350, 351, 352, 353, 354, 356, 357, 359, 361, 362, 363, 366, 367, 369, 372, 376, 377, 378, 379, 382, 383, 385, 394, 396, 397, 399, 401, 404, 405, 408, 410, 413, 417, 418, 419, 420, 426, 427, 437, 439, 441 or 446, wherein the numbering of the positions in the Fc region is that of the EU index as in Kabat in the Fc region, and wherein the antibody comprising the altered Fc region has enhanced antibody dependent cellular cytotoxicity (ADCC) and enhanced CD16 binding relative to a corresponding antibody comprising the parent Fc region.

2. The antibody of claim 1 wherein the altered Fc region comprises at least one amino acid substitution at a position corresponding to position 343, 345, 350, 351, 352, 353, 354, 356, 357, 359, 361, 362, 363, 366, 367, 369, 372, 376, 377, 378, 379, 382, 383, 385, 394, 396, 397, 399, 401, 404, 405, 408, 410, 413, 417, 418, 419, 420, 426, 427, 437, 439, 441 or 446.

3-5. (canceled)

6. The antibody of claim 1 wherein the altered Fc region has one or more of the following substitutions V240Q, P247C, P247F, P247H, P247I, P247L, P247M, P247N, P247T, P247V, S254W, H268D, H268E, E272D, K274D, K274G, K274H, K274P, K274S, K274T, K290D, K290T, Q295C, R301S, T307A, T307C, T307D, T307E, T307F, T307G, T307I, T307K, T307L, T307M, T307N, T307P, T307R, T307V, T307Y, V308P, D312L, D312T, K326C, K3261, K326L, K326T, K326V, A330F, K334I, K334P, K334T, K334V, P343A, P343R, E345W, T350H, T350K, T350W, L351R, P352W, P353R, S354K, S354R, E356K, E357R, T359R, N361W, N361Y, Q362M, V363Q, T366V, C367H, C367T, V369R, F372W, D376T, I377F, 1377H, I377K, 1377Q, I377R, I377Y, A378I, A378P, A378V, V379Y, E382G, E382H, E382Q, E382S, E382T, E382W, E382Y, S383K, G385R, G385W, T394F, T394Q, T394W, T394Y, P396I, P396Y, V397D, V397L, V397M, D399L, D401W, F404W, F405K, S408F, S408M, S408Y, L410P, D413R, W417Q, Q418Y, Q419W, G420W, S426W, V427R, T437W, K439P, L441P, and/or G446W.

7. The antibody of claim 1 wherein the at least one substitution in the altered Fc region is 240Q, 247C, 247H, 2471, 247L, 247M, 247N, 247T, 247V, 254W, 268D, 268E, 272D, 274D, 274G, 274H, 274P, 274S, 274T, 290D, 290T, 295C, 301S, 307A, 307C, 307D, 307E, 307F, 307G, 307I, 307K, 307L, 307M, 307N, 307P, 307R, 307V, 307Y, 308P, 312L, 312T, 326C, 326I, 326L, 326T, 326V, 330F, 334I, 334P, 334T, 334V, 343A, 343R, 345W, 350H, 350K, 350W, 351R, 352W, 353R, 354K, 354R, 356K, 357R, 359 R, 361W, 361Y, 362M, 363Q, 366V, 367H, 367T, 369R, 372W, 376T, 377F, 377H, 377K, 377Q, 377R, 377Y, 378I, 378P, 378V, 379Y, 382G, 382H, 382Q, 382S, 382T, 382W, 382Y, 383K, 385R, 385W, 394F, 394Q, 394W, 394Y, 396I, 396Y, 397D, 397L, 397M, 399L, 401W, 404W, 405K, 408F, 408M, 408Y, 410P, 413R, 417Q, 418Y, 419W, 420W, 426W, 427R, 437W, 439P, 441P, or 446W.

8. The antibody of claim 1 wherein the residue at the corresponding position in the parent Fc region is V240, P247, S254, H268, E272, K274, K290, Q295, R301, T307, V308, D312, K326, A330, K334, P343, E345, T350, L351, P352, P353, S354, E356, E357, T359, N361, Q362, V363, T366, C367, V369, F272, D376, I377, A378, V3379, E382, S383, G385, T394, P396, V397, D399, D401, F404, F405, S408, L410, D413, W417, Q418, Q419, G420, S426, V427, T437, K439, L441, or G446.

9. The antibody of claim 1 wherein the sequence of the altered Fc region is not a native Fc region sequence.

10. The antibody of claim 1 wherein the parent Fc region comprises a human IgG Fc region.

11. The antibody of claim 1 wherein the parent Fc region comprises an IgG1 Fc region.

12. The antibody of claim 1 wherein the substitution at position 334 is not an alanine substitution.

13. The antibody of claim 1 wherein the Fc region has 15 or fewer substitutions at a position corresponding to position 240, 247, 254, 268, 272, 274, 290, 295, 301, 307, 308, 312, 326, 330, 334, 343, 345, 350, 351, 352, 353, 354, 356, 357, 359, 361, 362, 363, 366, 367, 369, 372, 376, 377, 378, 379, 382, 383, 385, 394, 396, 397, 399, 401, 404, 405, 408, 410, 413, 417, 418, 419, 420, 426, 427, 437, 439, 441 or 446.

14. The antibody of claim 1 wherein the Fc region has 10 or fewer substitutions at a position corresponding to position 240, 247, 254, 268, 272, 274, 290, 295, 301, 307, 308, 312, 326, 330, 334, 343, 345, 350, 351, 352, 353, 354, 356, 357, 359, 361, 362, 363, 366, 367, 369, 372, 376, 377, 378, 379, 382, 383, 385, 394, 396, 397, 399, 401, 404, 405, 408, 410, 413, 417, 418, 419, 420, 426, 427, 437, 439, 441 or 446.

15. (canceled)

16. A composition comprising the antibody of claim 1 and a pharmaceutically acceptable carrier.

17. The composition of claim 16 which is sterile.

18. The composition of claim 16 which is a pharmaceutical composition.

19. A method of treating a mammal in need of said treatment, comprising administering an effective amount of the antibody of claim 1.

20. A method of treating a mammal in need of said treatment, comprising administering an effective amount of the composition of claim 16.

21. A method to provide an antibody with enhanced ADCC and enhanced CD16 binding, comprising:
a) providing a host cell which expresses an antibody comprising an altered Fc region, wherein the altered Fc region comprises at least one amino acid substitution relative to a parent IgG Fc region, wherein the at least one substitution is at a position corresponding to position 240, 247, 254, 268, 272, 274, 290, 295, 301, 307, 308, 312, 326, 330, 334, 343, 345, 350, 351, 352, 353, 354, 356, 357, 359, 361, 362, 363, 366, 367, 369, 372, 376, 377, 378, 379, 382, 383, 385, 394, 396, 397, 399, 401, 404, 405, 408, 410, 413, 417, 418, 419, 420, 426, 427, 437, 439, 441 or 446, wherein the numbering of the positions in the Fc region is that of the EU index as in Kabat in the Fc region; and
b) selecting a host cell that expresses an antibody comprising the altered Fc region which has enhanced ADCC and enhanced CD16 binding relative to a corresponding antibody comprising the parent Fc region.

22. The method of claim 21 further comprising isolating the antibody from the selected host cell.

23-26. (canceled)

27. The method of claim 21 wherein the altered Fc region has one or more of the following substitutions V240Q, P247C, P247F, P247H, P247I, P247L, P247M, P247N, P247T, P247V, S254W, H268D, H268E, E272D, K274D, K274G, K274H, K274P, K274S, K274T, K290D, K290T, Q295C, R301S, T307A, T307C, T307D, T307E, T307F, T307G, T307I, T307K, T307L, T307M, T307N, T307P, T307R, T307V, T307Y, V308P, D312L, D312T, K326C, K326I, K326L, K326T, K326V, A330F, K334I, K334P, K334T, K334V, P343A, P343R, E345W, T350H, T350K, T350W, L351R, P352W, P353R, S354K, S354R, E356K, E357R, T359R, N361W, N361Y, Q362M, V363Q, T366V, C367H, C367T, V369R, F372W, D376T, I377F, I377H, I377K, I377Q, I377R, I377Y, A378I, A378P, A378V, V379Y, E382G, E382H, E382Q, E382S, E382T, E382W, E382Y, S383K, G385R, G385W, T394F, T394Q, T394W, T394Y, P396I, P396Y, V397D, V397L, V397M, D399L, D401W, F404W, F405K, S408F, S408M, S408Y, L410P, D413R, W417Q, Q418Y, Q419W, G420W, S426W, V427R, T437W, K439P, L441P and/or G446W.

28. The method of claim 21 wherein the at least one substitution in the altered Fc region is 240Q, 247C, 247H, 247I, 247L, 247M, 247N, 247T, 247V, 254W, 268D, 268E, 272D, 274D, 274G, 274H, 274P, 274S, 274T, 290D, 290T, 295C, 301S, 307A, 307C, 307D, 307E, 307F, 307G, 307I, 307K, 307L, 307M, 307N, 307P, 307R, 307V, 307Y, 308P, 312L, 312T, 326C, 326I, 326L, 326T, 326V, 330F, 334I, 334P, 334T, 334V, 343A, 343R, 345W, 350H, 350K, 350W, 351R, 352W, 353R, 354K, 354R, 356K, 357R, 359R, 361W, 361Y, 362M, 363Q, 366V, 367H, 367T, 369R, 372W, 376T, 377F, 377H, 377K, 377Q, 377R, 377Y, 378I, 378P, 378V, 379Y, 382G, 382H, 382Q, 382S, 382T, 382W, 382Y, 383K, 385R, 385W, 394F, 394Q, 394W, 394Y, 396I, 396Y, 397D, 397L, 397M, 399L, 401W, 404W, 405K, 408F, 408M, 408Y, 410P, 413R, 417Q, 418Y, 419W, 420W, 426W, 427R, 437W, 439P, 441P, or 446W.

29. The method of claim 21 wherein the residue at the corresponding position in the parent Fc region is V240, P247, S254, H268, E272, K274, K290, Q295, R301, T307, V308, D312, K326, A330, K334, P343, E345, T350, L351, P352, P353, S354, E356, E357, T359, N361, Q362, V363, T366, C367, V369, F272, D376, I377, A378, V379, E382, S383, G385, T394, P396, V397, D399, D401, F404, F405, S408, L410, D413, W417, Q418, Q419, G420, S426, V427, T437, K439, L441, or G446.

30. The method of claim 21 wherein the Fc region has 15 or fewer substitutions at a position corresponding to position 240, 247, 254, 268, 272, 274, 290, 295, 301, 307, 308, 312, 326, 330, 334, 343, 345, 350, 351, 352, 353, 354, 356, 357, 359, 361, 362, 363, 366, 367, 369, 372, 376, 377, 378, 379, 382, 383, 385, 394, 396, 397, 399, 401, 404, 405, 408, 410, 413, 417, 418, 419, 420, 426, 427, 437, 439, 441 or 446.

31-33. (canceled)

34. A method to provide an antibody with enhanced ADCC and CD16 binding, comprising:
a) introducing to a parent IgG Fc region of an antibody one or more substitutions to yield an antibody comprising an altered Fc region, wherein at least one substitution in the altered Fc region is 240Q, 247C, 247H, 247I, 247L, 247M, 247N, 247T, 247V, 254W, 268D, 268E, 272D, 274D, 274G, 274H, 274P, 274S, 274T, 290D, 290T, 295C, 301S, 307A, 307C, 307D, 307E, 307F, 307G, 307I, 307K, 307L, 307M, 307N, 307P, 307R, 307V, 307Y, 308P, 312L, 312T, 326C, 326I, 326L, 326T, 326V, 330F, 334I, 334P, 334T, 334V, 343A, 343R, 345W, 350H, 350K, 350W, 351R, 352W, 353R, 354K, 354R, 356K, 357R, 359R, 361W, 361Y, 362M, 363Q, 366V, 367H, 367T, 369R, 372W, 376T, 377F, 377H, 377K, 377Q, 377R, 377Y, 378I, 378P, 378V, 379Y, 382G, 382H, 382Q, 382S, 382T, 382W, 382Y, 383K, 385R, 385W, 394F, 394Q, 394W, 394Y, 396I, 396Y, 397D, 397L, 397M, 399L, 401W, 404W, 405K, 408F, 408M, 408Y, 410P, 413R, 417Q, 418Y, 419W, 420W, 426W, 427R, 437W, 439P, 441P, or 446W, and wherein the numbering of the positions in the Fc region is that of the EU index as in Kabat in the Fc region; and
b) identifying an antibody comprising the altered Fc region which has enhanced ADCC and enhanced CD 16 binding.

35-74. (canceled)


SEQUENCE LISTING (15 sequences)

SEQ ID NO: 1 - protein, 227 aa, Artificial Sequence
  A synthetic Fc gamma receptor sequence.
MRAWIFFLLCLAGRALAMRAEDLPKAVVFLEPQWYRVLEKDSVTLKCQGAYSPEDNSTRWFHNESLISSQTSSYFIAAARVNNSGEYRCQTSLSTLSDPVQLEVHIGWLLLQAPRWVFKEEESIHLRCHSWKNTLLHKVTYLQNGKGRKYFHQNSDFYIPKATLKDSGSYFCRGLIGSKNVSSETVNITITQDLAVSSISSFFPPGYQTGTETSQVAPAASHHHHHH

SEQ ID NO: 2 - protein, 216 aa, Artificial Sequence
  A synthetic Fc gamma receptor sequence.
MRAWIFFLLCLAGRALAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPMTGTETSQVAPAASHHHHHH

SEQ ID NO: 3 - protein, 216 aa, Artificial Sequence
  A synthetic Fc gamma receptor sequence.
MRAWIFFLLCLAGRALAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMTGTETSQVAPAASHHHHHH

SEQ ID NO: 4 - protein, 213 aa, Artificial Sequence
  A synthetic Fc gamma receptor sequence.
MRAWIFFLLCLAGRALAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMTGTETSQVAPAASHHHHHH

SEQ ID NO: 5 - protein, 313 aa, Artificial Sequence
  A synthetic Fc gamma receptor sequence.
MRAWIFFLLCLAGRALAQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHTGTETSQVAPAASHHHHHH

SEQ ID NO: 6 - protein, 213 aa, Artificial Sequence
  A synthetic Fc gamma receptor sequence.
MRAWIFFLLCLAGRALAALPKAVVKLDPPWIQVLKEDMVTLMCEGTHNPGNSSTQWFHNGRSIRSQVQASYTFKATVNDSGEYRCQMEQTRLSDPVDLGVISDWLLLQTPQRVFLEGETITLRCHSWRNKLLNRISFFHNEKSVRYHHYKSNFSIPKANHSHSGDYYCKGSLGSTQHQSKPVTITVQDPATTSSTGTETSQVAPAASHHHHHH

SEQ ID NO: 7 - protein, 221 aa, Artificial Sequence
  A synthetic Fc gamma receptor sequence.
MRAWIFFLLCLAGRALAQAGLQKAVVNLDPKWVRVLEEDSVTLRCQGTFSPEDNSIKWFHNESLIPHQDANYVIQSARVKDSGMYRCQTALSTISDPVQLEVHMGWLLLQTTKWLFQEGDPIHLRCHSWQNRPVRKVTYLQNGKGKKYFHENSELLIPKATHNDSGSYFCRGLIGHNNKSSASFRISLGDPGSPSMFPPWHQTGTETSQVAPAASHHHHHH

SEQ ID NO: 8 - protein, 312 aa, Artificial Sequence
  A synthetic Fc gamma receptor sequence.
MRAWIFFLLCLAGRALAAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSGSGTGTETSQVAPAASHHHHHH

SEQ ID NO: 9 - protein, 208 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 10 - protein, 208 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQ

SEQ ID NO: 11 - protein, 17 aa, Artificial Sequence
  A synthetic signal peptide
MRAWIFFLLCLAGRALA

SEQ ID NO: 12 - protein, 233 aa, Homo sapiens
MGWSCIILFLVATATGVHSDIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 702 bp, Homo sapiens
atgggatggagctgtatcatcctgttcctcgtggccacagcaaccggtgtccacagcgacatccagatgacccagtctccaacctcactgtctgcatctgtaggagacagagtcaccatcacttgtcgggcgagtcagggtattagcagctggttaacctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtatgatagttaccctatcaccttcggccaagggacacgactggagattaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 14 - protein, 461 aa, Homo sapiens
MGWSCIILFLVATATGVHSQVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1386 bp, Homo sapiens
atgggatggagctgtatcatcctgttcctcgtggccacagcaaccggtgtccacagccaggtgcagctacagcagtggggcgcaggactgttgaagccttcggagaccctgtccctcacctgcgctgtctatggtgggtccttcagtgcttactactggagctggatccgccagcccccagggaaggggctggagtggattggggacatcaatcatggtggaggcaccaactacaacccgtccctcaagagtcgagtcaccatatcagtagacacgtccaagaaccagttctccctgaagctgaactctgtaaccgccgcggacacggctgtgtattactgtgcgagcctaactgcctactggggccagggaagcctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga